(19) United States
(12) Patent Application Publication
(10) Pub. No.: US 2023/0211290 A1
(43) Pub. Date: Jul. 6, 2023

(54) METHOD FOR SEPARATING BIOMASS FROM A SOLUTION COMPRISING BIOMASS AND AT LEAST ONE AROMA COMPOUND

(71) Applicant: BASF SE, Ludwigshafen am Rhein (DE)

(72) Inventors: Emiel Jan KAPPERT, Ludwigshafen am Rhein (DE); Jacek MALISZ, Ludwigshafen am Rhein (DE); Frederike CARSTENSEN, Ludwigshafen am Rhein (DE); Anne-Catrin HAILER, Ludwigshafen am Rhein (DE)

(21) Appl. No.: 18/008,749

(22) PCT Filed: Jun. 10, 2021
(86) PCT No.: PCT/EP2021/065686

§ 371 Date: Dec. 7, 2022

(30) Foreign Application Priority Data
Jun. 12, 2020 (EP) 20179760.2

(51) Int. Cl. B01D 61/14; B01D 61/22

(52) U.S. Cl. CPC B01D 61/146; B01D 61/22; B01D 2311/10; B01D 2311/18; B01D 2311/2626

(57) ABSTRACT

The present invention relates to a method for separating biomass from a solution comprising bi-omass and at least one aroma compound. comprising providing the solution comprising bio-mass and aroma compounds. lowering the pH value of the solution below 7 by adding at least one acid to the solution comprising biomass and the at least one aroma compound. adding an adsorbing agent to the solution comprising biomass and aroma compounds. and carrying out first membrane filtration so as to separate the biomass from the solution comprising the at least one aroma compound.

### TECHNICAL FIELD

The present invention relates to a method for separating biomass from a solution comprising biomass and at least one aroma compound.

### BACKGROUND

Attractive smells and flavours are liked by humans for many reasons, and compounds that are perceived as having an attractive smell or flavour are preferred. The cause of this attractive smell or flavour is the perception of aroma compounds, and in consequence aroma compounds have for a long time been of interest.

Natural sources of aroma compounds can be too limited for the demand, and the extraction and/or purification of the aroma compounds form their natural sources often is a laborious process.

Chemical synthesis of some aroma compounds is known, but not all desired aroma compounds can be made efficiently by chemical synthesis alone or require additional treatments. For example, the European patent application EP1081212 discloses a method of purification of furaneol after chemical synthesis using zeolite.

Production by chemical or biotechnological means has been used, but this provides new challenges in the isolation of the desired product.

For example, one or more aroma compound may be produced by means of fermentation providing a solution comprising biomass and at least one aroma compound. Such a solution may also be called fermentation broth. Many different aroma compounds can be produced by fermentation of microorganisms (see for an overview Vandamme and Soetart 2002)

One examples of aroma compounds produced by fermentation is vanillin. The U.S. Pat. Nos. 9,115,377 and 6,133,033 disclose the use of Amycolatopsis strains to produce vanillin. The patent application published as CN105132472 discloses the use of Streptomyces psammoticus, while CN1421523 discloses the use of Aspergillus niger. Yeasts have also been used, for example in the international patent application published as WO2007099230 or the Chinese patent application CN105219806. Also used for vanillin production has been Escherichia coli, which is a common organism in fermentation research and production, see for example the Korean patent KR10-1163542 or the recently published international patent applications WO2020/223417 and WO2020/223418.

Biomass separation from the fermentation broth from the aroma compound process is the first downstream processing step in the production of aroma compounds. The state-of-the-art technology for this step is centrifugation and or filter press, sometimes with the use of flocculants. However, microfiltration can also be employed and has several advantages in comparison to other separation technologies. To enable a genetically modified organism free product solution, microfiltration is the best option because it can completely retain all non-dissolved solids including genetically modified microorganisms. However, this has its own challenges in respect to the membrane performance and recovery, energy consumption to achieve the desired pressure and fluxes, proteins and colour compounds passing through and requiring costly subsequent steps, and product losses.

### SUMMARY

Membrane filtrations are often used to separate smaller molecules from larger ones in a solution. A membrane filtration followed by additional steps including an active carbon treatment to remove colour compounds has also been disclosed in the production of lactic acid in the US patent application published as US20110028759. Crossflow filtration is known for example from EP2583744 which discloses its use in particular in the production of enzymes.

The separation of the biomass after fermentative production of aroma compound is usually done at a pH value of 7 to 8, typically around pH 7, by means of an initial centrifugation or filter press and further centrifugations. Sometimes polymeric membranes are used instead. When membranes are used, however, the membrane performance is rather low and the permeate contains a lot of proteins and colour components, which have to be removed in the following steps leading to an elaborate downstream process, high product yield losses and some quality problems.

Typically, after these initial steps of biomass separation from fermentation broths, the next step carried out is an ultrafiltration completed typically with 10 kDa polyethersulfone membranes, yet not all proteins and polysaccharides can be separated by this. The ultrafiltration permeate is hence sent to an active carbon column to decolorize the solution and achieve an APHA value of below 1000. The decolourization in the active carbon column is a rather tedious process and it is often necessary to use around 14% weight/weight of active carbon in relation to the initial amount of fermentation broth. This step leads to high product losses and necessitates huge active carbon columns.

It was therefore an object of the invention to avoid the abovementioned disadvantages. In particular, a method should be provided that is suitable to enhance the performance of separating biomass from a solution comprising biomass and at least one aroma compound and to reduce the amount of proteins in and the colour of the filtration permeate.

According to the present invention, this object is solved by a method for separating biomass from a solution comprising biomass and at least one aroma compound, and optionally one or more disaccharides and/or one or more monosaccharides comprising:
providing the solution comprising biomass and aroma compounds,
lowering the pH value of the solution below 7, preferably below pH 5.5 or less by adding at least one acid to the solution comprising biomass and the at least one aroma compound,
adding an adsorbing agent to the solution comprising biomass and aroma compounds, and
carrying out a membrane filtration also called herein the first membrane filtration and typically being a microfiltration or ultrafiltration so as to separate the biomass from the solution comprising the at least one aroma compound. Preferably, the sequence of method steps is the one given in the previous sentence.

According to the method of the present invention, it was surprisingly found, that the membrane performance can be significantly increased, and removal of proteins can be significantly improved when the pH value of the solution is lowered below 7. Further, it was found that membrane performance increases further and the colour of the permeate can be significantly reduced to values below the required specification when an adsorbing agent is added to the solution before any membrane filtration. Also advantageously, the needed amount of adsorbing agent like active carbon is much lower as compared to the known methods, and also the required time for decolourization is much shorter than in known methods, when the membrane filtration is done after the pH value has been set to the desired target value below pH 7 and at least on adsorbing agent has been added.

Preferably, the adsorbing agent is active carbon. Active carbon, also known as activated carbon or activated charcoal, is a preferred adsorbing agent as it is of low cost, available in large quantities, easy to handle and safe to use in foodstuff.

It is beneficial to the methods of the invention that the pH value of the solution comprising biomass and one or more aroma compound, and optionally one or more disaccharide and/or one or more monosaccharide, is below pH 7.0 when the first membrane filtration is performed, and more preferably when the adsorbing agent is added. Hence, since pH values of fermentation broth are typically at or above pH 7.0, the pH value typically is lowered by the addition of at least one acid as needed to achieve the target pH value. In case the pH value of the solution comprising biomass and one or more aroma compound, and optionally one or more disaccharide and/or one or more monosaccharide is already below pH 7.0 at the start, at least one acid may be used for setting the pH value stably below pH 6.0 as needed. Also, preferably, the pH value of the solution is set to a pH value of 5.5 or below, before any membrane filtration is started. Preferably the pH value is adjusted to a target pH value in the range of 3.0 to 5.5, more preferably the range of 3.5 to 5, wherein the ranges given include the given numbers. In an even more preferred embodiment, the pH value of the solution is set to pH 3.5 or above, but not higher than pH 4.5 and most preferably the pH value is set to a value in the range of and including 4.0 to 4.5. To lower the pH value if needed, at least one acid is added to the solution. Said at least one acid is, more preferably, an acid selected from the group consisting of H₂SO₄, H₃PO₄, HCl, HNO₃ and CH₃CO₂H. Basically, any acid may be used. Nevertheless, these acids are usually easy to handle.

Said adsorbing agent, preferably active carbon, is typically added in an amount in the range of 0.25% to 3% by weight, preferably in the range of 0.5% to 3.0% by weight and more preferably in the range of 0.75% by weight to 2.5%, preferably 2.2% by weight and even more preferably in the range of 1.0% to 2.0% by weight. wherein the percentage values are on a weight of adsorbing agent per weight of solution basis. Alternatively amounts from and including 0.5 to 1.5% by weight may be used in the inventive methods. Thus, a rather small amount of said adsorbing agent, preferably active carbon, is sufficient to reduce the colour number below the upper bound specification, which is preferably 1000 APHA. This allows for significant reduction of active carbon consumption as well as for significant reduction of product losses in comparison to the active carbon column. In one embodiment one or more adsorbing agents are added in an amount suitable to bind—in increasing order of preference—at least 50%, 55%, 60%, 65%, 70%, 75%, 80%, 90%, 92%, 94%, 95% or more of the colour components and/or the protein in the starting solution comprising biomass and/or polysaccharides and/or proteins and/or nucleic acids like DNA or RNA that may be present. Further, said adsorbing agent, preferably active carbon, is typically added as a powder having a particle size distribution with a diameter d50 in the range of 2 μm to 25 μm, preferably in the range of 3 μm to 20 μm and more preferably in the range of 3 μm to 7 μm, and even more preferably in the range of 5 μm to 7 μm. The d50 value is determined with standard procedures. Particle sizes in this size range reduce the risk of abrasion of the membrane. Moreover, said adsorbing agent, preferably active carbon, is yet preferably added as a suspension of the powder in water. This facilitates handling of the adsorbing agent as the suspension of the powder may better mix with the suspension comprising biomass and the aroma compound. The adding said adsorbing agent, preferably active carbon, to the solution is, typically, carried out after adding the at least one acid to the solution. Unexpectedly, the colour reduction and protein reduction are much better, when the pH value is adjusted first and then the adsorbing agent or at least the majority of the adsorbing agent is added subsequently. It is possible to add said adsorbing agent, preferably active carbon, to the fermentation broth before adding the at least one acid to the solution.

In another variant, the pH value of the solution is lowered to 5.5, more preferably to 5.0 and even more preferably to 4.5 by the addition of at least one of the suitable acids, and then adsorbing agent, preferable active carbon, and further acid is added until the desired final pH value is achieved.

Also, some of the adsorbing agent may be added before any acid is added to lower the pH value, followed by the addition of more adsorbing agent after the pH value has been set to the target value below pH 7.0.

In one aspect of the inventions the one or more adsorbing agent is added as a suspension of the adsorbing agent powder in water.

Another aspect of the invention refers to said adsorbing agent, preferably active carbon, being added to the solution is carried out when the pH value of the solution is below 7, and while at least one acid continues to be added to the solution or after adding the at least one acid to the solution has been completed. Alternatively adding said adsorbing agent, preferably active carbon, to the solution is carried out before adding the at least one acid to the solution.

Preferably, said solution comprising biomass and one or more aroma compounds, and optionally one or more disaccharide and/or one or more monosaccharide typically is a fermentation broth, obtained by cultivation of one or more types of cells, preferably bacteria, plant or yeast cells, more preferably bacteria, even more preferably Escherichia coli, Amycolatopsis sp or Rhodobacter sphaeroides, in a cultivation medium, preferably a cultivation medium comprising at least one carbon source, at least one nitrogen source and inorganic nutrients. Thus, sufficient amounts of said aroma compound(s), may be produced with cost efficient methods. In one aspect of the invention the cell are genetically modified cells, for example genetically modified cells of Escherichia coli, Amycolatopsis sp or Rhodobacter sphaeroides.

Said microfiltration or ultrafiltration of the first membrane filtration step is typically carried out as cross-flow microfiltration or cross-flow ultrafiltration. Thus, the filtration efficiency may be enhanced. Said cross-flow microfiltration or cross-flow ultrafiltration includes a cross-flow speed above 0.2 m/s, preferably in the range of 0.5 m/s to 6.0 m/s, more preferably in the range of 2.0 m/s to 5.5 m/s and even more preferably in the range of 2.8 m/s to 4.5 m/s, and most preferably in the range of 3.0 m/s to 4.0 m/s if ceramic mono- and multi-channel elements are used. In another embodiment, the cross-flow speed is equal to or below 3.0 m/s. In case that a polymeric membrane is used for the first membrane filtration, cross-flow speeds of 2 m/s or less can be used; cross-flow speeds in the range of 0.5 m/s to 1.7 m/s are preferably used, but even crossflow speeds of 0.5 m/s or less may be used. In another preferred embodiment, the cross-flow speed is not more than 1.7 m/s, 1.6 m/s, 1.5 m/s, 1.4 m/s, 1.3 m/s, 1.2 m/s, 1.1 m/s or 1.0 m/s if a polymeric membrane is used. Thus, the filtration speed may be optimized when compared to a filtration process without including a pH value adjustment and addition of an adsorbing agent. By doing so, wear and tear on and/or energy consumption of the membrane filtration equipment can be reduced by operating at lower cross-flow speed compared to previously known methods, while resulting in good separation.

Said first membrane filtration, preferably a microfiltration or ultrafiltration is, typically, carried out at a temperature of the solution in the range of 4° C. to 55° C., preferably in the range of 10° C. to 50° C. and more preferably in the range of 30° C. to 40° C. Thus, the temperature during said filtration step may be the same as during fermentation which further improves the membrane performance and decreases viscosity of the solution comprising biomass and aroma compound. Yet, the first membrane filtration is, also preferably, carried out by means of a ceramic microfiltration membrane or ceramic ultrafiltration membrane having a pore size in the range of 20 nm to 800 nm, preferably in the range of 40 nm to 500 nm and more preferably in the range of 50 nm to 200 nm. It is also possible to use multi-layered membranes that are engineered to have improved abrasion resistance, e.g. 400 nm and 200 nm and 50 nm pore size layers of Al₂O₃. Thus, sufficient amounts of proteins and polysaccharides may be removed in order to comply with the desired specification. Also typically, first membrane filtration is carried out by means of a polymeric microfiltration membrane or polymeric ultrafiltration membrane having a cut-off above or equal to 4 kDa, preferably in the range of 10 kDa to 200 nm, more preferably in the range of 50 kDa to 200 nm and even more preferably equal to or above 100 kDa. In another preferred embodiment the cut-off is 100 nm or less. Thus, sufficient amounts of proteins and polysaccharides may be removed in order to comply with the desired specification.

The polymeric material of the polymeric microfiltration membrane or polymeric ultrafiltration membrane is, preferably, at least one polymeric material selected from the group consisting of: polyethersulfone, polysulfone, polypropylene, polyvinylidene fluoride, polyacrylonitrile, polyvinylidene fluoride. Modified polymeric materials can also be used, for example hydrophilized polyethersulfone.

The ceramic material of the ceramic microfiltration membrane or ceramic ultrafiltration membrane is, preferably, at least one ceramic material selected from the group consisting of: TiO₂, ZrO₂, SiC and Al₂O₃.

The first membrane filtration, preferably microfiltration or ultrafiltration is, typically, carried out after a predetermined time after the adsorbing agent, preferably active carbon, has been added to the solution. This allows to provide an adsorption time during which colour components are adsorbed. In one embodiment, said predetermined time is at least 2 min, preferably at least 10 min and more preferably at least 20 min. Thus, the adsorption of colour components typically is rather quick.

The method may, preferably, further comprise carrying out a second or further membrane filtration, preferably an ultrafiltration, using the solution essentially free of biomass obtained by the microfiltration or ultrafiltration of the first membrane filtration and comprising one or more aroma compounds, and optionally one or more disaccharides and/or one or more monosaccharides, preferably comprising the majority of these saccharides from the starting solution, e.g. the fermentation broth, that also comprised the biomass. Preferably, the second membrane filtration is done with the permeate of the first membrane filtration and with a membrane having a lower cut-off than the first membrane. Thus, an advantageous further processing of the permeate obtained by the first membrane filtration is realized. The second membrane filtration is, typically, an ultrafiltration carried out by means of an ultrafiltration membrane, which preferably is at least partially made of a polymeric material, and/or has a cut-off in the range of 1 kDa to 10 kDa, preferably in the range of 2 kDa to 10 kDa and more preferably in the range of 4 kDa to 5 kDa. Polymeric membranes typically offer the advantage over tight ceramic membranes that they are more robust and less expensive.

The second membrane filtration may be performed with a ceramic membrane of 1 to 25 kDa cut-off. In a further embodiment it is preferable that the membrane is at least partially made of a polymeric material. Said polymeric material is, more preferably, at least one polymeric material selected from the group consisting of: polyethersulfone, polysulfone, polyacrylonitrile, cellulose acetate. Said second membrane filtration is, typically, carried out after adjusting the temperature of the solution to temperatures of below 20, preferably at a temperature of the solution being in the range of 4° C. to 15° C., preferably in the range 8° C. to 13° C. and more preferably in the range 8° C. to 12° C.

In a preferred embodiment, the first membrane filtration employed in the inventive methods includes two or preferably three steps, also called sub-steps, as will be explained in further detail below. The first step includes a first diafiltration having a diafiltration factor DF (amount of diafiltration water=starting amount of fermentation broth x diafiltration factor) ranging from 0.5 or less to 3 or above. For example, for 2′FL comprising solutions it was advantageous to have a DF of 0.5 while for other aroma compound molecules values of 3 proved to be better if a concentration step was to follow. During diafiltration, the amount of water or a suitable aqueous solution added is identical to the amount of permeate discharged. In a batch wise diafiltration, the volume in the feed vessel is thus kept constant. The second step includes concentrating of the fermentation broth preferably with a factor 2 or more by stopping the feed of diafiltration water and the level will decrease down to the target value (target value=volume or mass at the beginning of the fermentation broth/concentrating factor). Optionally, the subsequent third step includes a second diafiltration. By means of these three steps of the first membrane filtration a lower dilution of the product within the permeate and an increased yield of 95% are realized.

By increasing the factor of the second diafiltration, the yield may even be further increased. However, the dilution of the product will also increase.

The permeate then typically is the combination of all solutions passing through the membrane in these three steps of the first membrane filtration. In a batch process each step produces a permeate fraction in a time-separated manner, that can be collected in one vessel for mixing, or processed separately. In a continuing process, each of the three steps produces a permeate fraction not in a time separated, and these fractions can be combined to form the permeate combined or treated separately if desired.

Optionally the first step of the first membrane filtration may be repeated one or more times, before the second step of concentration is done. Optionally, the second step may be performed, or it may be skipped if concentrating the solution is not desirable. This is useful when the fermentation broth has a high viscosity and or very high biomass content, for example.

Optionally the first step may be skipped and alternatively the second step is done without the first step, so that first a concentration of the fermentation broth is done while creating permeate, and then a diafiltration of the last step is done by feeding water or aqueous solutions to the solution comprising biomass and one or more aroma compounds.

A further embodiment is the inventive processing unit suitable to perform the methods of the invention, comprising the apparati for the pre-treatment, the first membrane filtration, the second membrane filtration and optionally for a reverse osmosis treatment or any further purification or concentration steps.

Further features and embodiments of the invention will be disclosed in more detail in the subsequent description, particularly in conjunction with the dependent claims. Therein the respective features may be realized in an isolated fashion as well as in any arbitrary feasible combination, as a skilled person will realize. The embodiments are schematically depicted in the figures. Therein, identical reference numbers in these figures refer to identical elements or functionally identical elements.

### DETAILED DESCRIPTION

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “particularly”, “more particularly”, “specifically”, “more specifically”, “typically”, “more typically”, “preferably”, “more preferably” or similar terms are used in conjunction with additional/alternative features, without restricting alternative possibilities. Thus, features introduced by these terms are additional/alternative features and are not intended to restrict the scope of the claims in any way. The invention may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

As used herein, the term “biomass” refers to the mass of biological material comprised in the solution. Typically, said biological material in accordance with the present invention are one or more types of prokaryotic or eukaryotic organisms, or parts thereof, such as cell walls, proteins, phospholipids, cell membranes, polynucleotides and other large organic compounds produced by the microorganism.

In a particular preferred embodiment, biomass refers to one or more biological organisms, more preferably the one or more organism is a bacterium or a fungal or a plant cell or non-human animal cell. In a preferred embodiment, the one or more organism is a bacterial cell selected from a) the group of Gram negative bacteria, such as Rhodobacter, Agrobacterium, Acetobacter, Paracoccus, Pseudomonas or Escherichia; b) a bacterial cell selected from the group of Gram positive bacteria, such as Arthrobacter, Bacillus, Corynebacterium, Brevibacterium, Amycolatopis; c) a fungal cell selected from the group of Aspergillus (for example Aspergillus niger), Blakeslea, Penicillium, Ceratocysis moniliformis, Trichoderma viride, T harzianum, Pycnoporus cinnabarinus, Phanerochaete chrysosporium, Phaffia (Xanthophyllomyces), Cryptococcus, Pichia, Saccharamoyces, Kluyveromyces, Yarrowia, Hansenula, Hyphozyma (for example H. roseoniger), Geotrichum klebahnii, Geotrichum fragrans, Sporidiobolus salmonicolor, and Williopsis saturnus var mrakii; or d) a transgenic plant or culture comprising transgenic plant cells, wherein the ocell is of a transgenic plant selected from Nicotiana spp, Cichorum intybus, Iacuca sativa, Mentha spp, Artemisia annua, tuber forming plants, oil crops and trees; e) or a transgenic mushroom or culture comprising transgenic mushroom cells, wherein the microorganism is selected from Schizophyllum, Agaricus and Pleurotisi. More preferred organisms are microorganism belonging to the genus Escherichia, Saccharomyces, Pichia, Amycolatopsis, Rhodobacter, and even more preferred those of the species E. coli, S. cerevisae, Rhodobacter sphaeroides or Amycolatopis sp., for example but not limited to Amycolatopsis mediterranei, for example the strain NCIM 5008, Streptomyces setonii, Streptomyces psammoticus, and for example but not limited to Amycolatopsis sp strains IM1390106, Zyl 926, ATCC39116, DSM 9991, 9992 or Zhp06.

More preferably, the said biomass comprises microorganisms, even more preferably genetically modified microorganisms, which are cultivated in a cultivation medium, preferably a cultivation medium comprising at least one carbon source, at least one nitrogen source and inorganic nutrients.

In a further embodiment, the methods of the invention are applied to separate aroma compounds, disaccharides and monosaccharides produced from macromolecular biomass, such as wood, straw, stalks and other plant material containing lignin, cellulose and/or starch, or from macromolecular biomass or animal or microbial origin, such as chitin containing substances, polysaccharides and the like from the remainders of said macromolecular biomass.

The easiest way to assess the success of separating the biomass and the aroma compound(s), disaccharide(s) and/or monosaccharide(s) is to monitor that the permeate of the first membrane filtration is optically clear. Unsuccessful separation will result in biomass being detected in the optical check of the permeate, and the presence of adsorbing agent like black active carbon in the permeate will also easily be detected in the optical check and indicate a leak or failure of the membrane filtration equipment.

As used herein, the term “aroma compound” refers to any substance that is an odorant, aroma, fragrance, or flavour, and preferably is a chemical compound that has a smell or odour, wherein the aroma compound has one glycosidic bond or no glycosidic bonds and is not a protein. Preferably an aroma compound in addition is an organic compound, typically an organic compound with less than 300 as a molecular weight. In one embodiment the aroma compound is to be understood to be a substance that is an odorant, aroma, fragrance, or flavour, and preferably is a chemical compound that has a smell or odour, has one glycosidic bond or no glycosidic bonds and is selected from those of the formula (C5H8)n, wherein n is an integer of 4 or higher, and is a compound other than a protein. In another embodiment n is an integer between and including 1 to 10. In another embodiment the aroma compound is not lactic acid or butyl butyrate. In a preferred embodiment the aroma compound as defined in this section is a polar aroma compound, even more preferably is selected from the list of Ambrox, Ambrox-1,4-diol, furaneol, benzoic acid, phenylethanol, raspberry ketone, pyrazines, sclareol, vanillin, vanillyl alcohol and vanilla glycoside, and yet even more preferably it is selected from vanillin, vanillyl alcohol and vanilla glycoside.

In one embodiment the methods of the invention refer to methods that produce a purified precursor of the final aroma compound rather than the finished aroma compound, wherein the precursor has been produced by fermentation and is present in the fermentation broth subjected to the inventive treatment. The methods of the invention and the processing units of the invention may as part of this invention be applied to such precursor molecules that after the purification by the inventive methods or in the inventive processing units are then transformed into the final aroma compound by one or more enzymatic and/or chemical steps. Therefore, in one embodiment any reference to an aroma compounds may be understood to refer to a precursor of said aroma compound.

In one embodiment aroma compound is a flavour compound. In another embodiment the aroma compound is a fragrance compound.

As used herein, the term “oligosaccharide” refers to a saccharide polymer containing a small number of typically three to ten of monosaccharides (simple sugars).

As used herein, the term “disaccharide” refers to a saccharide consisting of two monosaccharides, for example lactose that consists of a glucose and a galactose moiety, or saccharose that is made from one glucose and one fructose molecule.

As used herein, the term “monosaccharide” refers to a simple sugar, preferably a sugar molecule comprising 5 or 6 carbon atoms, for example glucose, fructose, galactose or fucose.

The term “adsorbing agent” as used herein refers to an element configured to provide the adhesion of atoms, ions or molecules from a gas, liquid or dissolved solid to a surface. The term “adhesion” refers to the tendency of dissimilar particles or surfaces to cling to one another. Preferably, the adsorbing agent is configured to provide adhesion for colour components. Preferably, the adsorbing is active carbon.

As used herein, the term “microfiltration” refers to a type of physical filtration process where a fluid comprising undesired particles, for example contaminated fluid is passed through a special pore-sized membrane to separate microorganisms and suspended particles from process liquid, particularly larger bacteria, yeast, and any solid particles. Microfiltration membranes haves a pore size of 0.1 μm to 10 μm. Thereby, such membranes have a cut-off for a molecular mass of more than 100000 kDa.

As used herein, the term “ultrafiltration” refers to a type of physical filtration process where a fluid comprising undesired particles, for example contaminated fluid is passed through a special pore-sized membrane to separate microorganisms and suspended particles from process liquid, particularly bacteria, macromolecules, proteins, larger viruses. Ultrafiltration membranes have typically a pore size of 2 nm to 100 nm and have a cut-off for a molecular mass of 2 kDa to 250000 kDa. The principles underlying ultrafiltration are not fundamentally different from those underlying microfiltration. Both of these methods separate based on size exclusion or particle retention but differ in their separation ability depending on the size of the particles.

According to the present inventive methods, first membrane filtration is carried out preferably by means of a polymeric microfiltration membrane or polymeric ultrafiltration membrane having a cut-off equal to or above 4 kDa, preferably in the range of 10 kDa to 200 nm, more preferably in the range of 50 kDa to 200 nm and even more preferably in the range of 50 kDa to 100 nm. Further, said second membrane filtration is preferably carried out by means of an ultrafiltration membrane having a cut-off in the range of 1 kDa to 10 kDa, preferably in the range of 2 kDa to 10 kDa and more preferably in the range of 4 kDa to 5 kDa.

The cut-off of a filtration membrane typically refers to retention of 90% of a solute of a given size or molecular mass, e.g. 90% of a globular protein with x kDa are retained by a membrane with a cut-off of x kDa. These cut-off values can be measured for example by the use of defined dextranes or polyethylene glycols and analyzing the retentate, the permeate and the original solution also called feed with a GPC gel permeation chromatography analyser using methods and parameters common in the art.

As used herein, the term “cross-flow filtration” refers to a type of filtration where the majority of the feed flow travels tangentially across the surface of the filter, rather than into the filter, at positive pressure relative to the permeate side. The principal advantage of this is that the filter cake which can blind the filters in other methods is not building up during the filtration process, increasing the length of time that a filter unit can be operational. It can be a continuous process, unlike batch-wise dead-end filtration. For large scale applications, a continuous process is preferable. This type of filtration is typically selected for feeds containing a high proportion of small particle size solids where the permeate is of most value because solid material can quickly block (blind) the filter surface with dead-end filtration. According to the present disclosure, said cross-flow microfiltration or cross-flow ultrafiltration includes a cross-flow speed in the range of (and including) 0.5 m/s to 6.0 m/s, preferably in the range of (and including) 2.0 m/s to 5.5 m/s and more preferably in the range of (and including) 2.2 m/s to 4.5 m/s and even more preferably in the range of (and including) 2.5 to 4.5 or in the range of (and including) 3.0 m/s to 4.5 m/s. In case of a membrane made of ceramics, the cross-flow speed may be higher than in case of a membrane made of a polymeric material depending on the respective geometry of the membrane. For example, in case of a flat polymeric membrane such as a polymeric membranes in flat sheet modules, the cross-flow speed is 0.5 m/s to 2.0 m/s and preferably 1.0 m/s to 1.7 m/s. and more preferably 1.0 to 1.5 m/s. Depending on the particular set-up and the particular solution comprising the biomass even cross-flow speeds of 1.0 m/s or less may be used in some cases, yet the filtration may turn into a dead end filtration when the cross-flow speeds are too low.

The term “cut-off” as used herein refers to the exclusion limit of a membrane which is usually specified in the form of MWCO, molecular weight cut off, with units in Dalton. It is defined as the minimum molecular weight of a solute, for example a globular protein that is retained to 90% by the membrane. The cut-off, depending on the method, can be converted to so-called D90, which is then expressed in a metric unit.

Passage or transmission should be understood to mean the movement of the compound through the membrane. In practice, passage is determined by calculating the ratio of permeate concentration to retentate concentration of the compound and is typically expressed as a percentage. In contrast, retention is the percentage of a compound that remains on the original side of a membrane during membrane filtration and is also expressed as a percentage, wherein ideally passage and retention for a given substance sum up to about 100%.

In a first step (FIG. 1, step S10), a solution comprising at least one aroma compound produced in a fermentative process is provided. Said at least one aroma compound comprises aroma compound, preferably a polar aroma compound. The at least one aroma compounds can be selected Ambrox, Ambrox-1,4-diol, furaneol, benzoic acid, phenylethanol, raspberry ketone, pyrazines, sclareol, vanillin, vanillyl alcohol and vanilla glycoside or a precursor thereof. Preferably, said solution comprises biomass as well as at least one aroma compound and is obtained by cultivation of one or more types of cells in a cultivation medium. Thus, said solution may also be called fermentation broth in a preferred embodiment. The cultivation medium is preferably a cultivation medium comprising at least one carbon source, at least one nitrogen source and inorganic nutrients. More preferably, the fermentation broth or solution comprising biomass and the at least one aroma compound is obtained by microbial fermentation, preferably aerobic microbial fermentation. A microorganism capable of producing the aroma compound may be a yeast or a bacterium or a plant cell or an animal cell, for example from the group consisting of the genera Escherichia, Klebsiella, Helicobacter, Bacillus, Lactobacillus, Streptococcus, Amycolatopsis, Rhodobacter, Lactococcus, Pichia, Saccharomyces and Kluyveromyces. The aqueous nutrient medium comprises at least one carbon source (e.g. glycerol or glucose) which is used by the microorganism for growth and/or for biosynthesis of the aroma compound. In addition, the nutrient medium also contains at least one nitrogen source, preferably in the form of yeast extract or an ammonium salt, e.g. ammonium sulphate, ammonium phosphate, ammonium citrate, ammonium hydroxide etc., which is necessary for the growth of the microorganisms. Other nutrients in the medium include e.g. one or several phosphate salts as phosphor source, sulphate salts as sulphur source, as well as other inorganic or organic salts providing e.g. Mg, Fe and other micronutrients to the microorganisms. In many cases, one or more vitamins, e.g. thiamine, has to be supplemented to the nutrient medium for optimum performance. The nutrient medium may optionally contain complex mixtures such as yeast extract or peptones. Such mixtures usually contain nitrogen-rich compounds such as amino acids as well as vitamins and some micronutrients.

The nutrients can be added to the medium at the beginning of the cultivation, and/or they can also be fed during the course of the process. Most often the carbon source(s) are added to the medium up to a defined, low concentration at the beginning of the cultivation. The carbon source(s) are then fed continuously or intermittently in order to control the growth rate and, hence, the oxygen demand of the microorganisms. Additional nitrogen source is usually obtained by the pH control with ammonia (see below). It is also possible to add other nutrients mentioned above during the course of the cultivation.

In some cases, a precursor compound is added to the medium, which is necessary for the biosynthesis of the aroma compound. The precursor compound may be added to the medium at the beginning of the cultivation, or it may be fed continuously or intermittently during the cultivation, or it may be added by a combination of initial addition and feeding.

The cells are cultivated under conditions that enable growth and biosynthesis of the aroma compound in a stirred tank bioreactor. For aerobic conditions a good oxygen supply in the range of 50 mmol O₂/(l*h) to 180 mmol O₂/(l*h) to the microbial cells is essential for growth and biosynthesis, hence in such a setting the cultivation medium is aerated and vigorously agitated in order to achieve a high rate of oxygen transfer into the liquid medium in case aerobic conditions are desired, and optionally, the air stream into the cultivation medium may be enriched by a stream of pure oxygen gas in order to increase the rate of oxygen transfer to the cells in the medium.

Anaerobic conditions are herein defined as conditions without any oxygen or in which substantially no oxygen is consumed by the cultured cells, in particular a microorganism, and usually corresponds to an oxygen consumption of less than 5 mmol/l·h, preferably to an oxygen consumption of less than 2.5 mmol/l·h, or more preferably less than 1 mmol/l·h.

Oxygen-limited conditions are defined as conditions in which the oxygen consumption is limited by the oxygen transfer from the gas to the liquid. The lower limit for oxygen-limited conditions is determined by the upper limit for anaerobic conditions, i.e. usually at least 1 mmol/l·h, and in particular at least 2.5 mmol/l·h, or at least 5 mmol/l·h. Some organisms are grown in micro-aerob conditions as a special variant of oxygen limited conditions. In such cases substantially more than 5 mmol/l·h are used, but not enough to arrive at full aerobic conditions. The upper limit for oxygen-limited conditions is determined by the lower limit for aerobic conditions, i.e. less than 100 mmol/l·h, less than 50 mmol/l·h, less than 20 mmol/l·h, or less than to 10 mmol/l·h, depending on the organisms and the set-up like growth temperature, growth stage and the like.

For example, the organism may be kept at dissolved oxygen levels between 10% and 40%, for example at 35% and then after some time reduced to 15%.

Aerobic conditions are conditions in which a sufficient level of oxygen for unrestricted growth is dissolved in the medium, able to support a rate of oxygen consumption of at least 10 mmol/l·h, more preferably more than 20 mmol/l·h, even more preferably more than 50 mmol/l·h, and most preferably more than 100 mmol/l·h.

Whether conditions are aerobic, anaerobic or oxygen-limited is dependent on the conditions under which the method is carried out, in particular by the amount and composition of ingoing gas flow, the actual mixing/mass transfer properties of the equipment used, the type of micro-organism used and the micro-organism density.

Generally, the temperature is at least 0° C., in particular at least 15° C., more in particular at least 20° C. A desired maximum temperature depends upon the enzymes producing the aroma compound and the host cell used. Depending on the cells and/or the enzymes producing the aroma compound used, the temperature is 70° or less, preferably 50° C. or less, more preferably 40° C. or less, in particular 37° C. or less. Organisms like Thermus thermophilus have a temperature optimum for growth between 49° C. and 72° C., Escherichia coli of about 37° C., and many fungal microorganisms like yeasts and bacterial microorganisms like Rhodobacter sphaeroides have temperature optima around 30° C. In case of a fermentative process, the incubation conditions can be chosen within wide limits as long as the cells show sufficient activity and/or growth. This includes pH ranges, temperature ranges and aerobic, oxygen-limited and/or anaerobic conditions.

In one embodiment the cultivation is carried out at 24° C. to 41° C., preferably 28° C. to 40° C., more preferably at 30° C. or more. Depending on the organism used a temperature of around 30° C. or in the range of 32° C. to 39° C. is used. The pH value is set at 6.2 to 7.2, preferably by automatic addition of NH₃ (gaseous or as an aqueous solution of NH₄OH).

In some cases, the biosynthesis of the aroma compound needs to be induced by addition of a chemical compound, e.g. Isopropyl β-D-1-thiogalactopyranoside (IPTG) for example as in the European patent application published as EP 2 379 708. The inducer compound may be added to the medium at the beginning of the cultivation, or it may be fed continuously or intermittently during the cultivation, or it may be added by a combination of initial addition and feeding.

Subsequently, the method of the invention proceeds to the adjustment of the pH value in a second step (FIG. 1, step S12). In said step, typically the pH value of the solution is lowered to below pH 7.0 by adding at least one acid to the solution comprising biomass and the at least one aroma compound. Said at least one acid is an acid selected from the group consisting of H₂SO₄, H₃PO₄, HCl, HNO₃ (preferably not in concentrated form) and CH₃CO₂H, or any other acid considered safe in production of food or feed; preferably the acid is selected from the group consisting of H₂SO₄, H₃PO₄, HCl and CH₃CO₂H. A mix of these acids may be used in one embodiment instead of a single of these acids. The pH value of the solution is adjusted to a target pH value preferably in the range of 3.0 to 5.5, more preferably in the range of 3.5 to 5 and even more preferably in the range of 4.0 to 4.5, such as 4.0 or 4.1.

Further, in another embodiment of the method of the invention, if the solution comprising biomass and the at least one aroma compound, and optionally at least one disaccharide or at least one monosaccharide already has a pH value below 7, preferably below pH 5.5, more preferably equal to or below pH 5.0 and even more preferably equal to or below pH 4.5, the addition of any of these acids is optional, and step S12 may be skipped and the methods of the invention for such solutions continues with Step S14.

However, even if the pH value to start with is below pH 7.0, or even below pH 5.5 or pH 5.0 or even 4.5, lowering the pH will lead to further benefits like higher flux, less pressure to be applied and even better removal of colour and proteins in the subsequent steps, and also even more reduced membrane fouling. Hence in a preferred embodiment the pH value is adjusted to a target pH value preferably in the range of 3.0 to 5.5, more preferably in the range of 3.5 to 5 and even more preferably in the range of 4.0 to 4.5, such as 4.0 or 4.1—even if the pH value at the start of this step is already below pH 7.0, or even below pH 6.0.

In some circumstances, for example for organisms or biomass treatments requiring very low pH values for the generation of the aroma compound, the pH value of the solution comprising biomass and at least one aroma compound may be lower than pH 4.0, maybe even lower than pH 3.0. In this case adjusting the pH to a target pH value preferably in the range of 3.0 to 5.5, more preferably in the range of 3.5 to 5 and even more preferably in the range of 4.0 to 4.5, such as 4.0 or 4.1, may involve increasing the pH value by addition of pH increasing substances prior to the next step. This will balance performance and wear and tear on the equipment.

The method then proceeds to the next step (FIG. 1, S14). In said step, one or more adsorbing agent is added to the solution comprising biomass and the at least one aroma compound. Preferably, the adsorbing agent is active carbon. Said adsorbing agent, preferably active carbon, is added in an amount in the range of 0.5% to 3% by weight, preferably in the range of 0.6% to 2.5% by weight and more preferably in the range of 0.7% to 2.0% by weight, such as 1.5%. In this respect, it has to be noted that the smaller the particles of the adsorbing agent are, the better the adsorption characteristics are. Said adsorbing agent, preferably active carbon, is added as a powder having a particle size distribution with a diameter d50 in the range of 2 μm to 25 μm, preferably in the range of 3 μm to 20 μm and more preferably in the range of 3 μm to 7 μm such as 5 μm. More preferably, said adsorbing agent, preferably active carbon, is added as a suspension of the powder in water. Preferably, adding said adsorbing agent, preferably active carbon, to the solution is carried out after adding the at least one acid to the solution. Alternatively, adding said adsorbing agent, preferably active carbon, to the solution may be carried out before adding the at least one acid to the solution. With other words, the order of steps S12 and S14 may be changed and the order thereof is not fixed. Yet if the order is first setting of the pH below 7 to the desired pH value and then adding one or more adsorbing agents, preferably active carbon, will generate the best results with respect to protein removal and decolourization. In a preferred embodiment addition of the at least one acid antedates the addition of the at least one adsorbing agent, preferably active carbon.

The steps S12 and S14 may be performed in any order or repeated, for example S12 then S14 then S12 again, or S14 then S12 then S14 In a preferred embodiment of the methods of the invention, the steps S12 and S14 are both performed and in the order S12 followed by S14.

In between the step of adding one or more adsorbing agents to the solution and the subsequent steps like the first membrane filtration, an optional incubation step (S15) sufficient for the one or more adsorbing agents to adsorb at least 60%, preferably at least 70%, more preferably at least 75% and even more preferably at least 80, 85, 86, 87, 88, 89, 90, 91, 92, 93, 94, 95, 96, 97, 98 or 99% of the colour components in the solution may be used in the methods of the invention. In some cases, the incubation step may be short and for example not required as a fully separate step. For example, the transition time from adding the one or more adsorbing agent to the start of the first membrane filtration may already be enough time for the adsorbing agents to adsorb the undesired compounds sufficiently, but in some circumstances extra time before the start of the first membrane filtration will further improve the result.

The method then proceeds with first membrane filtration, preferably a micro- or ultrafiltration in a further step (FIG. 1, step S16) including a time suitable for the adhesion of colour components to the one or more adsorbing agents before the separation. The first membrane filtration is carried out so as to separate at least to a large extent the biomass and the one or more adsorbing agents from the solution comprising the at least one aroma compound, and optionally at least one disaccharide and/or at least one monosaccharide, and by this removing the biomass and also reducing the colour components and protein in the resulting solution also called permeate comprising the one or more aroma compounds, and optionally the one or more disaccharides and/or monosaccharides. Basically, step S16 includes microfiltration or ultrafiltration. However, as there is a smooth transition between microfiltration and ultrafiltration and both can be used by the skilled artisan to the purpose of separating biomass, adsorbing agent and protein on one side and the permeate containing the bulk of the desired one or more aroma compounds, and optionally one or more disaccharides and/or one or more monosaccharides, on the other side. The filtration in step S16 may also be an ultrafiltration as an alternative to microfiltration. Said microfiltration or ultrafiltration is preferably carried out as cross-flow microfiltration or cross-flow ultrafiltration to improve membrane performance and reduce membrane abrasion. The details of the filtration in step S16 will be explained below. Said cross-flow microfiltration or cross-flow ultrafiltration includes a cross-flow speed in the range of 0.5 m/s to 6.0 m/s, preferably in the range of 2.0 m/s to 5.5 m/s and more preferably in the range of 3.0 m/s to 4.5 m/s, such as 4.0 m/s. In one embodiment the cross-flow speed is equal to or below 3.0 m/s, preferably between and including 1.0 and 2.0. One advantageous of the inventive method, use and the processing units of the invention is that lower cross-flow speeds can be used to achieve good separation preferably of protein components of the solution from any aroma compounds, disaccharides or monosaccharides. Thus, energy and equipment cost can be reduced, wear and tear on equipment and abrasion of the filtration membrane are also reduced. Said first membrane filtration, preferably microfiltration or ultrafiltration, is carried out at a temperature of the solution in the range of 8° C. to 55° C., preferably in the range of 10° C. to 50° C. and more preferably in the range of 30° C. to 40° C., such as 38° C. Said microfiltration or ultrafiltration is carried out by means of a ceramic or polymeric microfiltration membrane or ceramic ultrafiltration membrane having a pore size in the range of 20 nm to 800 nm, preferably in the range of 40 nm to 500 nm and more preferably in the range of 50 nm to 200 nm, such as 100 nm. Said ceramic material in the methods or processing units of the invention is or has at least one layer of at least one ceramic material selected from the group consisting of: Titanium dioxide (TiO₂), Zirconium dioxide (ZrO₂), Silicon carbide (SiC) and Aluminium oxide (Al₂O₃). Alternatively, said microfiltration or ultrafiltration is carried out by means of a polymeric microfiltration membrane or polymeric ultrafiltration membrane having a cut-off in the range of 10 kDa to 200 nm, preferably in the range of 50 kDa to 200 nm and more preferably in the range of 50 kDa to 100 nm. Said polymeric material is at least one polymeric material selected from the group consisting of: polyethersulfone, polysulfone, polypropylene, polyvinylidene fluoride, polyacrylonitrile, polyvinylidene fluoride. Said first membrane filtration, preferably microfiltration or ultrafiltration, is carried out after a predetermined time after the adsorbing agent, preferably active carbon, has been added to the solution. Thus, ensures adhesion of colour components. Typically, the time needed for mixing of the solution with the added adsorbing agent until a homogenous distribution of the adsorbing agent, preferably active carbon, in the solution has been reached may suffice to allow for the adhesion of the colour components, yet a longer incubation time can be used to maximize this.

In one embodiment, said predetermined time is at least 2 min, preferably at least 10 min and more preferably at least 20 min such as 25 min or 30 min.

In a preferred embodiment the transmission of the one or more aroma compounds in this step is greater than 50%, preferably at least 55% and more preferably at least 60%, even more preferably greater than 70%, 75%, 80%, 85%, 90%, 95% or 98%. Retention of the one or more aroma compounds preferably is below 30%, more preferably below 25% and even more preferably below 20%, 15%, 10%, 8%, 6%, 4% or 2%.

The aim of said first membrane filtration, preferably microfiltration or ultrafiltration, is a purification of the solution comprising at least one aroma compound by removal of cells, cell debris, particles and other components of large size.

By performing the preceding steps before the first membrane filtration, a higher flux can be achieved, while less pressure difference across the membrane length is needed. The transition of proteins and colour compounds across the membrane into the permeate is effectively reduced and the yield of the aroma compound in the permeate of the first membrane filtration is increased compared to a membrane filtration without the preceding steps according to the invention.

In one embodiment, the method of the invention typically then proceeds with a second membrane filtration step (FIG. 1, step S18). Preferably an ultrafiltration of the solution comprising aroma compounds obtained by the first membrane filtration of step S16 is carried out. In other words, an ultrafiltration of the permeate derived from the first membrane filtration in step S16 is carried out. Preferably, said second membrane filtration, preferably ultrafiltration, is carried out by means of an ultrafiltration membrane having a cut-off in the range of 1.5 kDa to 10 kDa, preferably in the range of 2 kDa to 10 kDa and more preferably in the range of 4 kDa to 5 kDa. In a particularly preferred embodiment, membranes with a cut-off of 4 kDa or 5 kDa are suitable. Said ultrafiltration membrane is at least partially made of a polymeric material. Said polymeric material is at least one polymeric material selected from the group consisting of: polyethersulfone, polyacrylonitrile, cellulose acetate. Typically, the solution comprising aroma compound obtained by the first membrane filtration is brought to a temperature of below 20° C. before and preferably maintained a temperature of below 20° C. during said second membrane filtration. One aspect of the invention refers to said second membrane filtration, preferably ultrafiltration, is carried out at a temperature of the solution being in the range of 5° C. to 15° C., preferably in the range 8° C. to 13° C. and more preferably in the range 8° C. to 12° C., such as 10° C. Performing the second membrane filtration after the previous steps of the inventive methods results in an increased yield of the aroma compound after the second membrane filtration, reduced transmission of proteins, lower colour of the permeate of the second membrane filtration, higher concentration factor of the feed and higher average flux when compared to a method comprising a first and a second membrane filtration, but lacking the inventive pre-treatment.

FIG. 2 displays the sequence of steps of the inventive methods with the time suitable for the adhesion of the majority of the colour components to the one or more adsorbing agents before the separation shown as a separate step (S15 in FIG. 2). Such a separate incubation step may be favorable when long times for sufficient adhesion of the undesired compounds to the adsorbing agent are required. Further, FIG. 2 depicts for the first membrane filtration (which is S16 in FIG. 1) as a step with three parts or sub-steps; the three sub-steps of first membrane filtration being first diafiltration, concentrating and then optionally a second diafiltration. These are shown as S16/1, S16/2 and S16/3, respectively, in FIG. 2. The other steps are as in FIG. 1. Further, an additional, optional step of revere osmosis is shown as S20.

These sub-steps of step S16 are useful for example when an absorbance of the one or more aroma compounds of interest to the active carbon is observed. BY adjusting the sub-steps of step S16 the desired aroma compound can be released from the active carbon to a very large extend and hence is also found in the permeate of the first membrane filtration despite the active carbon pre-treatment leading to a temporary absorbance of the desired aroma compound to the active carbon.

In one embodiment of the invention, the method includes after the second membrane filtration step, another step (S20) in which the permeate of the second membrane filtration step S18 is subjected to a reverse osmosis treatment to largely remove any remaining solid particles.

For the avoidance of doubt, any reference to the protein content of the solution or the permeate or retentate is referring to free protein in the solution/permeate/retentate, i.e. the protein found extracellularly and not the protein contained in the biomass if any. During fermentation and also subsequent handling and membrane filtrations, protein may be liberated from biomass and then be considered free protein.

For the avoidance of doubt, any reference to the at least one aroma compound, in the solution or the permeate or retentate is referring to the at least one aroma compound not bound in the solution/permeate/retentate, i.e. the at least one aroma compound, found extracellularly and not the ones contained in the biomass if any. During fermentation and also subsequent handling and membrane filtrations, the at least one aroma compound may be liberated from biomass and then be considered not bound aroma compound in the solution.

In a preferred embodiment, the step of carrying out first membrane filtration, preferably a microfiltration or ultrafiltration, so as to separate the biomass from the solution comprising the at least one aroma compound, is to be understood as a purification step for separating the biomass and optionally cell debris, particles and other components of large size from the at least one aroma compound wherein the majority of the at least one aroma compound is found in the permeate of the first membrane filtration following the separation of biomass.

In one embodiment the invention refers to a method for separating biomass from a solution comprising biomass and at least one aroma compound, and optionally at least one disaccharide and/or at least one monosaccharide, comprising the steps of
a. providing the solution comprising biomass and at least one aroma compound, and optionally at least one disaccharide and/or at least one monosaccharide,
b. lowering the pH value of the solution below 7.0 by adding at least one acid to the solution comprising biomass and the at least one aroma compound, and optionally at least one disaccharide and/or at least one monosaccharide,
c. adding one or more adsorbing agents to the solution comprising biomass and at least aroma compound, and optionally at least one disaccharide and/or at least one monosaccharide,
d. Optionally an incubation step sufficient for the one or more adsorbing agents to bind at least 60%, preferably at least 70%, more preferably at least 80% and even more preferably at least 85, 86, 87, 88, 89, 90, 91, 92, 93, 94, 95, 96, 97, 98 or 99% of the colour components in the solution; and
e. carrying out first membrane filtration, preferably a microfiltration or ultrafiltration, so as to separate the biomass from the solution comprising the at least one aroma compound, and optionally at least one disaccharide and/or at least one monosaccharide.

In a preferred embodiment, the first membrane filtration is followed by an ultrafiltration as second membrane filtration, then optionally followed by a nanofiltration, ion exchange and/or reverse osmosis.

In one aspect of the invention, the first membrane filtration is followed by an ultrafiltration, then optionally followed by a reverse osmosis to concentrate the permeate of the ultrafiltration further and remove further solid particles.

A further aspect of the invention refers to a processing unit comprising i) a solution containing biomass, at least one adsorbing agent, preferably active carbon, and at least one aroma compound and optionally at least one disaccharide and/or at least one monosaccharide, wherein the pH value of the solution is below 7.0; ii.) a first filtration membrane, preferably a microfiltration or an ultrafiltration membrane; iii.) means to carry out a first membrane filtration across said first filtration membrane, preferably a microfiltration or ultrafiltration, to generate a permeate containing the bulk of the aroma compounds, disaccharides and or monosaccharides at a temperature of the solution in the range of 8° C. to 55° C., preferably in the range of 10° C. to 50° C. and more preferably in the range of 30° C. to 40° C.; and iv.) means to separate the permeate of the first membrane filtration from the solution as described in i). above; and further optionally comprising one or more of the following features: v.) means to transport said permeate of the first membrane filtration to a second filtration membrane, vi.) means to adjust the temperature of the permeate to a temperature below 20° C., vii.) a second filtration membrane, preferably an ultrafiltration membrane, viii.) means to carry out a second membrane filtration, preferably a ultrafiltration, at a temperature below 20° C., and ix.) means to keep separate the permeate of the second membrane filtration from the permeate of the first membrane filtration; and optionally x) means to carry out a reverse osmosis treatment of the permeate of the second membrane filtration;

wherein the surfaces of the parts of the processing unit that are in contact with the solution or any of the permeates are tolerant to pH values as low as pH 3.5 and optionally are made of material suitable for the production of food.

Another aspect of the invention refers to a processing unit comprising:

i. a vessel holding a solution containing biomass and at least one aroma compound and optionally at least one disaccharide and/or at least one monosaccharide
ii. means to adjust the temperature of said solution to a temperature between 5° C. and 70° C., preferably in the range of 8° C. to 55° C., more preferably in the range of 10° C. to 50° C. and even more preferably in the range of 30° C. to 40° C.;
iii. a measuring system to measure the pH value of the solution in the vessel;
iv. means to set the pH value of the solution to a value below 7.0, preferably a target pH value lower than 5.5, wherein preferably the means to set the pH are suitable for the addition of at last one acid,
v. Means to add at least one adsorbing agent, preferably active carbon, to the solution,
vi. Means to generate an essentially homogenous distribution of the adsorbing agent in the solution,
vii. a first filtration membrane, preferably a microfiltration or an ultrafiltration membrane,
viii. means to carry out with the help of said first filtration membrane a first membrane filtration, preferably a microfiltration or ultrafiltration, of the solution with a pH value below 7.0 and containing biomass, at least one adsorbing agent, preferably active carbon and at least one aroma compound and optionally at least one disaccharide and/or at least one monosaccharide, and wherein the means are suitable to generate a permeate containing the bulk of the aroma compounds, disaccharides and/or monosaccharides, and
ix. means to collect, transport and optionally store the permeate of the first membrane filtration wherein the permeate is generated from the solution with a pH value below 7.0 and containing biomass, at least one adsorbing agent, preferably active carbon and at least one aroma compound and optionally at least one disaccharide and/or at least one monosaccharide, and further optionally comprising:
x. means to transport said permeate of the first membrane filtration to a second filtration membrane,
xi. means to adjust the temperature of the first permeate to a temperature below 20° C.,
xii. a second filtration membrane, preferably an ultrafiltration membrane,
xiii. means to carry out a second membrane filtration, preferably an ultrafiltration, at a temperature below 20° C., and
xiv. means to separate the permeate of the second membrane filtration from the permeate of the first membrane filtration,
xv. optionally means to carry out a reverse osmosis treatment of the permeate of the second membrane filtration;
wherein the surfaces of the parts of the processing unit that are in contact with the solution or any of the permeates are tolerant to pH values as low as pH 3.5 and optionally are made of material suitable for the production of food.

Another aspect of the invention refers to a method for reducing wear and tear on and/or energy consumption of membrane filtration equipment used in the separation of biomass from a solution comprising at least one aroma compound, and optionally at least one disaccharide and/or at least one monosaccharide, wherein the method comprises these steps:
a. providing the solution comprising biomass and aroma compound(s),
b. adjusting the pH value of the solution to a pH value in the range of 3.0 to 5.5, preferably the range of 3.5 to 5 and more preferably the range of 4.0 to 4.5; for example if the pH value is pH 7.0 or higher lowering the pH value of the solution below 7 by adding at least one acid to the solution comprising biomass and comprising at least one aroma compound, and optionally at least one disaccharide and/or at least one monosaccharide,
c. adding one or more adsorbing agents, preferably active carbon, to the solution comprising biomass and aroma compound(s), preferably in an amount in the range of 0.5% to 3% by weight, preferably in the range of 0.75% to 2.5% by weight or from and including 0.5% to and including 1.5% by weight and more preferably in the range of 1.0% to 2.0% by weight,
d. Optionally as required an incubation step sufficient for the one or more adsorbing agents to bind the colour components in the solution, and
e. carrying out first membrane filtration so as to separate the biomass from the solution comprising the comprising at least one aroma compound, and optionally at least one disaccharide and/or at least one monosaccharide preferably at cross-flow speeds of no more than 3 m/s.

For the easier storage and transport, it is often desirable to have the aroma compound in solid form rather than in solution. Hence, in a preferred embodiment the inventive method as a final step has the removal of the desired aroma compound(s) like Ambrox, Ambrox-1,4-diol, furaneol, benzoic acid, phenylethanol, raspberry ketone, pyrazines, sclareol, vanillin, vanillyl alcohol and vanilla glycoside from the solution. This may be done by crystallisation for example, but not limited to crystallisation, with the help of one or more solvents such as but not limited to short chain alcohols (e.g. methanol, ethanol, propanol, butanol) and/or organic acids, preferably food-grade organic acids such as but not limited to acetic acid and/or propionic acid. Alternatively, solidification may be achieved in said final step of the inventive method by spray-drying or any other method for removal of water or solvent from the desired aroma compound to a suitable dryness of the aroma compound. Also, such steps of removing the aroma compound from the solution may be employed before said final step. For example the inventive method encompasses steps of crystallisation or spray drying followed by re-dissolving the aroma compound to create a new solution, optional other purification steps or repetitions of the removal from solution and re-dissolving of the aroma compound to form a new solution and then as a final step removal from the solution again.

Sometimes the desired at least one aroma compound is present as a liquid at room temperature and preferably used as liquid, for example oil and the like; hence in an alternative embodiment, a solidification step is not needed for such aroma compound(s). A solvent removal may be done for example.

This patent application claims the priority of the patent application EP20179760.2. Incorporated by reference are those embodiments of the priority application that are listed as further embodiments on page 16, line 24 to page 22, line 22.

In one embodiment, the fermentation broth is a fermentation broth from a two-phase fermentation process for example as disclosed in the international patent application published as WO2015002528, and optionally the fermentation broth used in the current invention is one or more water-immiscible organic solvents used in the two-phase fermentation process.

Summarizing, the present invention includes the following embodiments, wherein these include the specific combinations of embodiments as indicated by the respective interdependencies defined therein.

### FIGURES

FIG. 1 shows a block diagram of a method for separating biomass from a solution comprising biomass and at least one aroma compound according to the present invention.

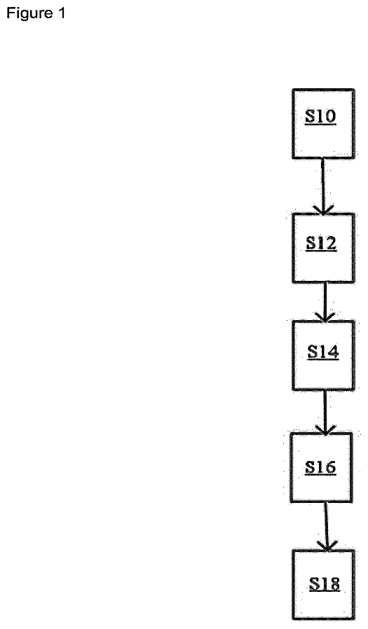

FIG. 2 displays in a block diagram the sequence of steps of the inventive methods when a separate incubation step S15 for the adsorbing agent and the three sub-steps S16/1 to S16/3 as explained above.

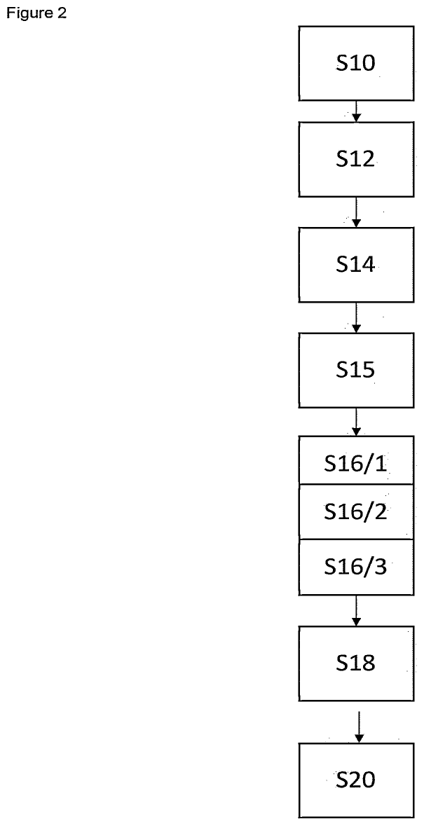

FIG. 3: Comparison of membrane flux as a function of DF and feed quality (with and without pre-treatment with 1.0% (w/w) active carbon). Circles represent without pre-treatment, squares with pre-treatment.

FIG. 4: Trends of UF membrane flux as a function of CF and feed quality (with and without pre-treatment with 1.0% (w/w) active carbon). Circles represent without pre-treatment, squares with pre-treatment.

FIG. 5: Comparison of membrane flux as a function of DF and feed quality (with and without pre-treatment with 1.5% w/w active carbon. Circles represent without pre-treatment, squares with pre-treatment.

FIG. 6: Comparison of UF membrane flux as a function of CF and feed quality (with and without pre-treatment). Circles represent without pre-treatment, squares with pre-treatment.

FIG. 7: Comparison of UF membrane flux as a function of relative permeate amount and feed AC concentration. Circles represent Trial 3A (0.7% (w/w) of AC), squares Trial 3B (1.5% (w/w) of AC)

### EXAMPLES

The method according to the present invention will be described in further detail below. Whatsoever, the Examples shall not be construed as limiting the scope of the invention.

### Generalized Example

A fermentation broth as a complex solution comprising biomass and at least one aroma compound is being prepared by standard methods. The pH value thereof is lowered to 4±0.1 by means of adding 10% sulphuric acid. Thereafter, a 30% suspension of active carbon Carbopal Gn-P-F (Donau Carbon GmbH, Gwinnerstrafle 27-33, 60388 Frankfurt am Main, Germany), which is food safe, is added and stirred for 20 min.

The thus prepared solution is supplied to the process apparatus, a semi-automatic MF lab unit from Sartorius AG, Otto-Brenner-Str. 20, 37079 Goettingen, Germany, modified for the purpose, and heated to 37° C. in a circulating manner with closed permeate. For separation purposes, the process apparatus includes a ceramic mono channel element (from Atech Innovations GmbH, Gladbeck, Germany) having an outer diameter of 10 mm, an inner diameter of 6 mm, a length of 1.2 m and a membrane made of Al₂O₃ having a pore size of 50 nm. As soon as the circulation of the solution is running and the solution comprises the target temperature of 37° C., the discharging of the permeate is started and the control of the trans membrane pressure is activated.

After terminating of the first membrane filtration, the process apparatus is stopped, the concentrate is disposed, and the process apparatus is being cleaned. Cleaning is carried out by means of 0.5% to 1% NaOH at a temperature of 50° C. to 80° C., wherein the NaOH is subsequently removed by purging.

In one embodiment, the first membrane filtration of the inventive methods includes three steps as will be explained in further detail below. The first step includes a first diafiltration having a factor of 0.5 (amount of diafiltration water=starting amount of fermentation broth x diafiltration factor). During diafiltration, the amount of water added is identical to the amount of permeate discharged. The first step is a continuing step and the volume in the feed vessel is thus kept constant. The second step includes concentrating of the fermentation broth with the factor 2 by stopping the feed of diafiltration water and the level will decrease down to the target value (target value=volume or mass at the beginning of the fermentation broth/concentrating factor). Subsequently, the third step includes a second diafiltration. The permeates collected during these three steps are typically combined to form the permeate. By means of these three steps a lower dilution of the product within the permeate and an increased yield are realized. By increasing the factor of the second diafiltration, the yield may even be increased.

The following analytical methods are been carried out.
HPLC or GC or GC-MS for the determination of the product, i.e. aroma compounds, and secondary components
Drying balances for measuring the dry content (DC)
APHA for measuring the colour using standard methods, for example DIN EN ISO 6271
Bradford protein assay for measuring the concentration of protein.

Hereinafter, the following abbreviations are used:
AC=Active Carbon
UF=Ultrafiltration
DP=Pressure drop along the module (p_{feed}-pᵣₑₜₑₙₜₐₜₑ)
Flux=Permeate flow rate per m² and hour (l/m² h)
Cross-flow velocity=linear speed of the suspension in membrane channels (m/s)
Membrane load=amount of permeate produced by 1 m² of membrane area (m³/m²)

Further, regarding the liquid separation, the following symbols and explanations are used.

| Symbol | Meaning | Unit | Definition |
|---|---|---|---|
| C | Concentration | wt-%, g/L | |
| CF | Concentration | — | m_{R, t=0}/m_{R} |
| | factor | | |
| DF | Diafiltration | — | m_{P}/m_{R, t=0} |
| | factor | | |
| J | Flux | LMH = L m⁻² | |
| | | h⁻¹ | |
| m | Mass | kg | |
| P | Pressure | bar | |
| R | Retention | — | 1 − cₚₑᵣₘₑₐₜₑ/cᵣₑₜₑₙₜₐₜₑ |
| TMP | Trans-membrane | bar | (p_{feed} + pᵣₑₜₑₙₜₐₜₑ)/2 − |
| | pressure | | pₚₑᵣₘₑₐₜₑ |

In the following, examples of the inventive methods with vanillin as the aroma compound are explained in detail to exemplify the invention without limiting it.

Common elements to the purification experiments for Vanillin described in the following: Separation of biomass from dissolved components was completed in a batch dynamic cross flow lab unit (called MF), running fully automatically, allowing for temperature, TMP, circulation flow control as well as automatic dosing of DF medium to the feed vessel. Permeate flow rate was measured by a scale. As a separation barrier ceramic 50 nm Al₂O₃ membrane from Atech Innovations GmbH (abrasion resistant three layers membrane 50/200/400 nm), mono tube 10/6, with 1.2 m length, having effective membrane area of 0.0223 m2. Only after reaching pre-defined crossflow velocity and predefined filtration temperature, TMP controlled permeate flow was activated.

Before and after each trial membrane performance (flux) was measured during rinsing with water at 25° C. Rinsing was running for 30 minutes. After each trial the unit was at first rinsed with deionized water, drained, and then cleaned with 0.5% Ultrasil® 125 (membrane cleaner from Ecolab) at 68-70° C. for 30 minutes. After draining the cleaner solution, the unit was rinsed and drained three times to remove completely cleaner solution and finally flux was measured again with deionized water at 25° C.

Permeates from MF were then treated in a lab UF unit to separate macro molecules: proteins, polysaccharides, RNA, DNA, etc. by means of polymeric Ultrafiltration membrane. In the trials the 4 kD PES membrane UH004 from Microdyn-Nadir was used.

During UF trials only concentration of MF permeate was applied (no diafiltration). The concentration was running until the minimum retentate amount was left in the unit or the permeate flux was reduced below 5 kg/m2 h.

UF tests were completed in a flat cell unit having three round membrane cells connected serially, having 0.00793 m2 membrane area. The feed pressure was provided by pressure control of N2 in the feed vessel, the cross-flow velocity was provided by a gear pump. Permeate flow was measured by a scale.

Similar membrane regeneration procedure as for ceramic membrane was used for polymeric membrane. Rinsing was completed at 30° C. and membrane cleaning at 50° C. for the same time.

Samples of fermentation broth, retentate and permeate were analyzed for proteins (according to Bradford method), color (APHA on PerkinElmer Lambda 35), dry content by drying samples (Mettler-Toledo HX204, Moisture Analyzer), and vanillin with GC method (see table A below for details). Samples of fermentation broth and MF retentate were filtrated with 0.2-micron filter, to ensure solid free samples.

TABLE A
GC conditions

| Column: | Fused silica, DB-1 ms UI | |
|---|---|---|
| Length: | 30 | m |
| Internal diameter: | 0.25 | mm |
| Film thickness: | 0.25 | μm |
| Carrier gas: | Nitrogen | |
| Column Flow/Head pressure | 1 mL/min (constant flow) | |
| Split ratio: | 15:1 | |
| Septum purge: | 3 | mL/min |
| Oven temperature: | 50° C., 5 min isothermal | |
| | 50° C. to 240° C., 5K/min | |
| Injector temperature: | 250° | C. |
| Detector temperature: | 300° | C. |
| Injection volume: | 1 | μl |

A fermentation broth of a standard lab strain of E. coli cells grown under typical conditions for a time that corresponds the known times for E. coli for growth through the growth phase and into the bioconversion phase. To simulate a fermentation broth of E. coli that has produced the aroma compound vanillin, vanillin was added to the fermentation broth to a concentration of around 10 g/I and gently mixed.

This vanillin containing fermentation broth was used for the following experiments.

### Example No. 1

An aliquot of the fermentation broth batch was divided in two portions. One part (Trial 1A) was directly diafiltrated with DF (diafiltration factor) of 4 using deionized water. The second part of fermentation broth (Trial 1B) was acidified with 20% H2SO4 to reach pH 4.5 and then so much of AC active carbon powder Carbopal Gn-P-F was added to obtain 1.0% (g/g) AC concentration. After that this suspension was identically diafiltrated with DF of 4 using deionized water. MF conditions with fermentation broth without pretreatment (Trial 1A):

T=35° C.

TMP=1 bar

Cross-flow velocity 4 m/s (4001/h)

MF conditions with fermentation broth with pretreatment (Trial 1B):

T=35° C.

TMP=1 bar

Cross-flow velocity 3.5 m/s (3501/h)

UF conditions with both permeates

T=10° C.

TMP=10 bar

Cross-flow velocity 1.5 m/s

Results from Separation of Biomass from Dissolved Components by a First Membrane Filtration by Microfiltration (MF)

Table 1 presents the performance data of biomass separation with ceramic membrane for fermentation broth without pretreatment (Trial 1A) and with pretreatment (Trial 1B). Membrane performance is 2.5 times higher, when fermentation broth was pretreated with pH adjustment and with addition of 1% of AC powder even though the cross-flow velocity is reduced. Due to this reduction in the crossflow velocity, the pressure difference (DP) along 1.2 m long membrane is reduced by 25%. This significantly reduces electrical energy demand by an industrial unit.

TABLE 1
Performance data

| | Flux | DP | cross-flow |
|---|---|---|---|
| Trial | [kg/m²h] | [bar] | [m/s] |
| A | 124 | 1.01 | 4.0 |
| B | 305 | 0.85 | 3.5 |

Table 2 presents membrane performance, measured with deionized water, before each trial, during rinsing after the trial, and after membrane regeneration. The results indicate that the membrane performance was easy re-established, slightly better after the trial with 1% AC.

TABLE 2
Membrane regeneration data

| | | | | | Flux- |
|---|---|---|---|---|---|
| | Flux [kg/m²h] | | | Regeneration | Improvement |
| Trial | before trial | after trial | after CIP | [%] | [%] |
| 1A | 509 | 150 | 477 | 94 | |
| 1B | 477 | 251 | 532 | 112 | 67 |

Table 3 (see following page) presents mass balance for both trials for APHA, protein, dry content of dissolved components, and vanillin. The data show clearly that pretreatment of fermentation broth significantly reduces color components not only in permeate but also in feed and retentate. Concentration of proteins is reduced by a factor of >5 and their retention is significantly higher in comparison to the trial without pretreatment.

Whereas protein amount in permeate in trial 1A is similar to the amount in feed, the amount of proteins is reduced by factor 3 in permeate from trial 1B.

The analyzed vanillin concentration in feed is significantly lower in feed in pretreated fermentation broth, indicating that vanillin partly adsorbs on AC. However, during diafiltration step it was possible to wash it out, so that similar amount of vanillin could be found in permeate.

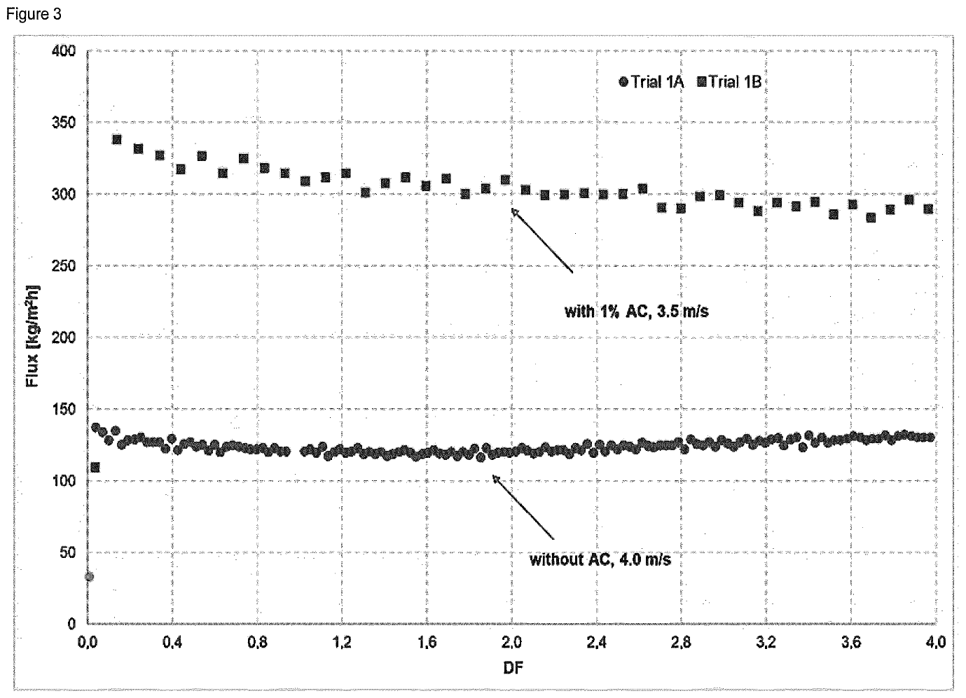

FIG. 3 presents flux trends as a function of diafiltration factor for both trials. The trends indicate that even with the higher solids load (1% AC) the membrane performance in trial 1B is significantly higher. A stable filtration performance was reached with or without active carbon.

Results from Concentration of the Permeates of the First Membrane Filtration with Ultrafiltration (UF) as Second Membrane Filtration

Table 4 presents reached concentration factors and average flux during the trial. The results clearly indicate the advantages of pretreatment of the fermentation broth. Concentration factor is by factor 7 higher as well as the average membrane performance is almost three times higher when fermentation broth was pretreated.

TABLE 4
Performance data

| | | Flux |
|---|---|---|
| Trial | CF | [kg/m²h] |
| 1A | 4.6 | 10 |
| 1B | 34.8 | 28 |

Table 5 provides membrane regeneration data, indicating that the chosen membrane can be easily regenerated even it was strongly fouled as in trial 1A.

TABLE 5
Membrane regeneration data

| | Flux [kg/m²h] | | Regeneration |
|---|---|---|---|
| UF trial | before trial | after trial | [%] |
| 1A | 72 | 66 | 92 |
| 1B | 62 | 59 | 95 |

TABLE 3
First membrane filtration: Mass balance of both trials

| | | Mass | | Protein | | Rₚᵣₒₜₑᵢₙ | DC (dissolved) | | Vanillin | | | R_{Vanillin} |
|---|---|---|---|---|---|---|---|---|---|---|---|---|
| Trial | Stream | [g] | APHA | [mg/l] | [g] | [%] | [%] | [g] | [g/l] | [g] | Yield | [%] |
| 1A | Feed | 1611.0 | 1988 | 198 | 0.32 | | 6.48 | 104.4 | 9.0 | 14.5 | | |
| | Permeate | 6456.5 | 418 | 63 | 0.41 | 68 | 1.49 | 96.2 | 2.0 | 12.9 | | |
| | Retentate | 1606.9 | 78 | 23 | 0.04 | | 0.13 | 2.1 | 0.6 | 1.0 | 0.07 | 32 |
| 1B | Feed | 1210.7 | 735 | 114 | 0.14 | | 6.31 | 76.4 | 5.0 | 6.1 | | |
| | Permeate | 4859.4 | 160 | 11 | 0.05 | 90 | 1.39 | 67.5 | 2.2 | 10.7 | | |
| | Retentate | 1207.7 | 12 | 17 | 0.02 | | 0.14 | 1.7 | 0.2 | 0.2 | 0.04 | 20 |

Yield is given as % (w/w); R_{Protein} is the retention of proteins, R_{Vanillin} is the retention of Vanillin; same applies to table 6.

TABLE 6
Second membrane filtration: Mass balance of both trials

| | | Mass | | Protein | | Rₚᵣₒₜₑᵢₙ | DC (dissolved) | | Vanillin | | Yield | Rᵥₐₙᵢₗₗᵢₙ |
|---|---|---|---|---|---|---|---|---|---|---|---|---|
| Trial | Stream | [g] | APHA | [mg/l] | [g] | [%] | [%] | [g] | [g/l] | [g] | [%] | [%] |
| 1A | Feed | 2712.2 | 418 | 63 | 0.17 | | 1.59 | 4.3 | 2.0 | 5.4 | | |
| | Permeate | 2116.6 | 245 | 60 | 0.13 | 5 | 1.20 | 2.5 | 2.0 | 4.2 | 78 | |
| | Retentate | 595.6 | 1187 | 181 | 0.11 | | 3.09 | 1.8 | 3.0 | 1.8 | 33 | 27 |
| 1B | Feed | 4454.0 | 160 | 11 | 0.05 | | 1.53 | 6.8 | 2.0 | 8.9 | | |
| | Permeate | 4326.1 | 115 | 4 | 0.02 | 65 | 1.24 | 5.4 | 2.0 | 8.7 | 97 | |
| | Retentate | 127.9 | 1903 | 152 | 0.02 | | 7.81 | 1.0 | 2.0 | 0.3 | 3 | 0 |

Table 6 (see previous page) presents the mass balance for both trials for APHA, dry content, proteins and vanillin. In permeate after pretreatment (trial 1B) APHA is reduced by factor 2, concentration of proteins by factor 25 in comparison to trial 1A (without pretreatment). Dry content in trial 1B is slightly higher, indicating that dissolved components pass the membrane with lower retention, because of significantly lower protein concentration and its fouling tendency. During that investigation period, polysaccharides were not analyzed but they surely play important role by membrane fouling. Vanillin recovery in trial 1B was with 97% significantly higher than in trial 1A with 78%. Its retention in trial 1B was 0%, vs. 27 in trial 1A.

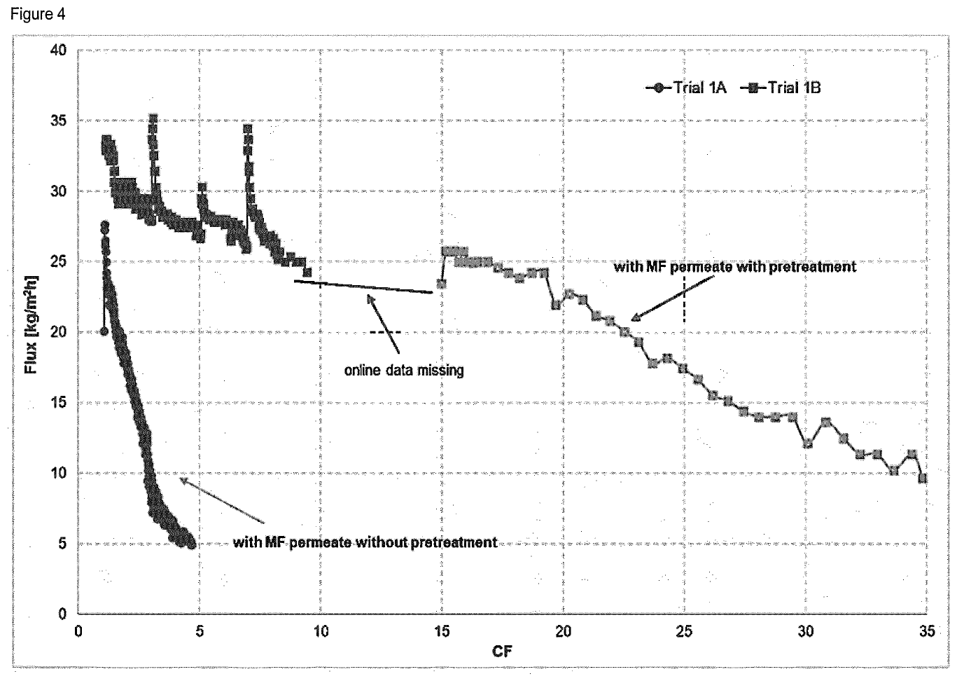

FIG. 4 provides trends of flux for both trials as a function of concentration factor. Membrane performance in trial 1B was still twice as high after CF of 34.8 as in trial 1A after CF of 4.6. Because of online data collection problem in trial 1B a part of flux values is missing. Because the operation volume of the UF unit was limited to 1.5 liters, the unit had to be stopped and refilled with feed solution. This was done three times during the 1B trial. After each restart the flux increased but relatively quick reached the previous values.

Subsequent Reverse Osmosis

Permeates from the UF trials 1A and 1B were concentrated by reverse osmosis in stirred test cells using different reverse osmosis membranes. Two types of Membranes (AlfaLaval R099 and Microdyn-Nadir ACM2) were used for both samples

Both experiments started with feeds with approximately same solid contents (1.24% for the experiments with active carbon, 1.20% for the experiments without active carbon). The results indicate that the reverse osmosis shows a better performance for broths that were treated with pH adjustment and active carbon. If the samples were pre-treated with pH adjustment and active carbon before the first membrane filtration, the solid retention and the Vanillin retention were higher for both membranes, and fluxes were continuously improved.

### Summary: Advantages of Pre-Treatment

Biomass separation by pre-treatment and first membrane filtration:
2.5-time higher flux
Lower pressure difference through membrane length
5.5-time less proteins in permeate
2.5-time lower APHA
Higher vanillin yield in permeate

UF separation of macromolecules:
7-time higher concentration factor of the feed
2.8-time higher average flux
2-times lower APHA
25-time less proteins in permeate
Significantly higher vanillin yield (97% vs 78%)

Reverse Osmosis
Better solid retention
Better Vanillin retention

### Example No. 2

A new fermentation broth batch was again divided in two portions after the addition of vanillin to around 10 g/l. The trials were conducted identical as in example 1 except a different amount of activated charcoal was used compared to example 1. One part (Trial 2A) was directly diafiltrated, with DF of 4 using deionized water. The second part of fermentation broth (Trial 2B) was acidified with 20% H2SO4 to reach pH 4.5 and then so much of AC active carbon powder Carbopal Gn-P-F was added to obtain 1.5% of its concentration. After that the pretreated fermentation broth was diafiltrated with DF of 4 using deionized water.

MF conditions with fermentation broth without pretreatment (Trial 2A):

T=35° C.

TMP=1 bar

Cross-flow velocity 4 m/s (4001/h)

MF conditions with fermentation broth with pretreatment (Trial 2B):

T=35° C.

TMP=1 bar

Cross-flow velocity 3.5 m/s (3501/h)

UF conditions with both MF permeates

T=10° C.

TMP=10 bar

Cross-flow velocity 1.5 m/s

Results from Separation of Biomass from Dissolved Components (MF)

Table 7 presents the performance data of biomass separation with ceramic membrane for fermentation broth without pretreatment (Trial 2A) and with pretreatment (Trial 2B). Membrane performance is 2.5 times higher, when fermentation broth was pretreated with pH adjustment and with addition of 1% of AC powder even though the cross-flow velocity is reduced, identical to the trail 1B in the first example.

TABLE 7
Performance data

| | Flux | DP | cross-flow |
|---|---|---|---|
| Trial | [kg/m²h] | [bar] | [m/s] |
| 2A | 171 | 1.00 | 4.0 |
| 2B | 286 | 0.85 | 3.5 |

Table 8 presents membrane performance, measured with deionized water, before each trial, during rinsing after the trial, and after membrane regeneration. The results indicate that the membrane performance was easy re-established.

TABLE 8
Membrane regeneration data

| | | | | | Flux- |
|---|---|---|---|---|---|
| | Flux [kg/m²h] | | | Regeneration | Improvement |
| Trial | before trial | after trial | after CIP | [%] | [%] |
| 2A | 532 | 182 | 501 | 94 | |
| 2B | 501 | 228 | 473 | 94 | 25 |

Table 9 (see following page) presents mass balance for both trials for APHA, protein, dry content of dissolved components, and vanillin. The data show clearly that pretreatment of fermentation broth with pH adjustment to 4.5 and the addition of 1.5% AC significantly reduces color components not only in permeate but also in feed and retentate. In permeate from pretreated fermentation broth: APHA is reduced by factor of 3, concentration of proteins is reduced by a factor of 35 and their retention is significantly higher. However, Vanillin concentration in feed is much lower in feed in pretreated fermentation broth, indicating that vanillin partly adsorbs on AC. However, during diafiltration step it was possible to wash it so that 15% more vanillin could be recovered, and its concentration was 2.5 times lower in retentate. Vanillin yield and retention for trial 2B were calculated with the dissolved vanillin concentration in the feed, and that is why they do not depicture the reality.

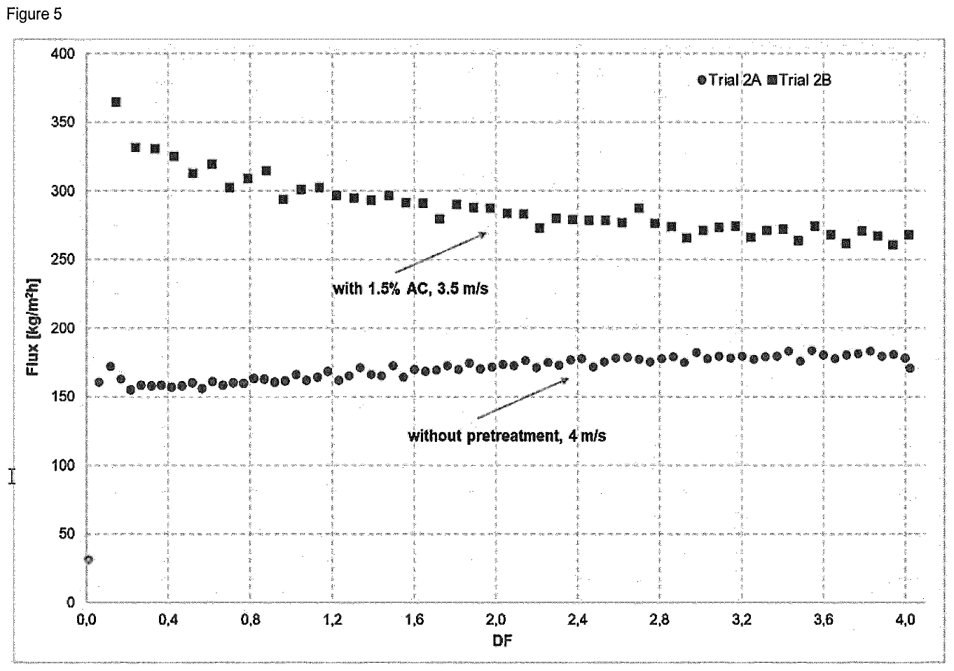

FIG. 5 presents flux trends as a function of diafiltration factor for both trials. The trends indicate that even by higher solids load (1.5% AC) the membrane performance in trial 2B is significantly higher. In both cases a stable filtration performance was reached. Similar trends as in the first example were measured.

Results from Concentration of MF Permeates with UF

Table 10 presents reached concentration factors and average flux during the trial. The results clearly indicate the advantages of pretreatment of the fermentation broth. Concentration factor is by factor 13 higher as well as the membrane performance is five times higher when fermentation broth was pretreated.

TABLE 10
Performance data

| | | Flux |
|---|---|---|
| Trial | CF | [kg/m²h] |
| 2A | 2.9 | 6 |
| 2B | 39.0 | 31 |

TABLE 9
First membrane filtration: Mass balance of both trials

| | | Mass | | Protein | | Rₚᵣₒₜₑᵢₙ | DC (dissolved) | | Vanillin | | | Rᵥₐₙᵢₗₗᵢₙ |
|---|---|---|---|---|---|---|---|---|---|---|---|---|
| Trial | Stream | [g] | APHA | [mg/l] | [g] | [%] | [%] | [g] | [g/l] | [g] | Yield | [%] |
| 2A | Feed | 1180.4 | 1259 | 226 | 0.27 | | 6.90 | 81.4 | 9.0 | 10.6 | | |
| | Permeate | 4746.2 | 375 | 46 | 0.22 | 80 | 1.48 | 70.2 | 2.0 | 9.5 | | |
| | Retentate | 1183.1 | 73 | 3 | 0.01 | | 0.09 | 4.3 | 0.5 | 2.4 | 0.22 | 62 |
| 2B | Feed | 1292.9 | 415 | 33 | 0.04 | | 6.26 | 80.9 | 3.0 | 3.9 | | |
| | Permeate | 5263.8 | 126 | 3 | 0.02 | 91 | 1.37 | 72.1 | 2.1 | 11.1 | | |
| | Retentate | 1266.3 | 14 | 11 | 0.06 | | 0.11 | 5.7 | 0.2 | 1.0 | 0.27 | 67 |

Yield is given as % (w/w); R_{Protein} is the retention of proteins, R_{Vanillin} is the retention of Vanillin; same applies to table 12

TABLE 12
Second membrane filtration: Mass balance of both trials

| | | Mass | | Protein | | Rₚᵣₒₜₑᵢₙ | DC (dissolved) | | Vanillin | | Yield | R_{Vanillin} |
|---|---|---|---|---|---|---|---|---|---|---|---|---|
| Trial | Stream | [g] | APHA | [mg/l] | [g] | [%] | [%] | [g] | [g/l] | [g] | [%] | [%] |
| 2A | Feed | 2109.0 | 375 | 46 | 0.10 | | 1.63 | 3.4 | 2.0 | 4.2 | | |
| | Permeate | 1384.6 | 195 | 38 | 0.05 | 17 | 1.16 | 1.6 | 3.0 | 2.8 | 66 | |
| | Retentate | 724.4 | 724 | 122 | 0.09 | | 2.48 | 1.8 | 3.0 | 2.2 | 52 | 38.0 |
| 2B | Feed | 4921.8 | 126 | 3 | 0.01 | | 1.48 | 7.3 | 2.0 | 9.8 | | |
| | Permeate | 4768.7 | 98 | 2 | 0.01 | 33 | 1.29 | 6.2 | 2.0 | 9.5 | 97 | |
| | Retentate | 153.1 | 1662 | 140 | 0.02 | | 7.87 | 1.2 | 2 | 0.3 | 3 | 5.0 |

Table 11 provides membrane regeneration data, indicating that the chosen membrane can be easily regenerated even it was strongly fouled as in trial 2A.

TABLE 11
Membrane regeneration data

| | Flux [kg/m²h] | | Regeneration |
|---|---|---|---|
| UF trial | before trial | after trial | [%] |
| 2A | 73 | 71 | 97 |
| 2B | 67 | 65 | 97 |

Table 12 (see previous page) presents the mass balance for both trials for APHA, dry content, proteins and vanillin. In permeate after pretreatment (trial 2B) APHA is reduced by factor 2, concentration of proteins by factor 19 in comparison to trial 2A (without pretreatment). Dry content in trial 2B is slightly higher, indicating that dissolved components pass the membrane with lower retention. Vanillin recovery in trial 2B was with 97% significantly higher than in trial 2A with 66%. Its retention in trial 2B was 5%, vs. 33 in trial 2A.

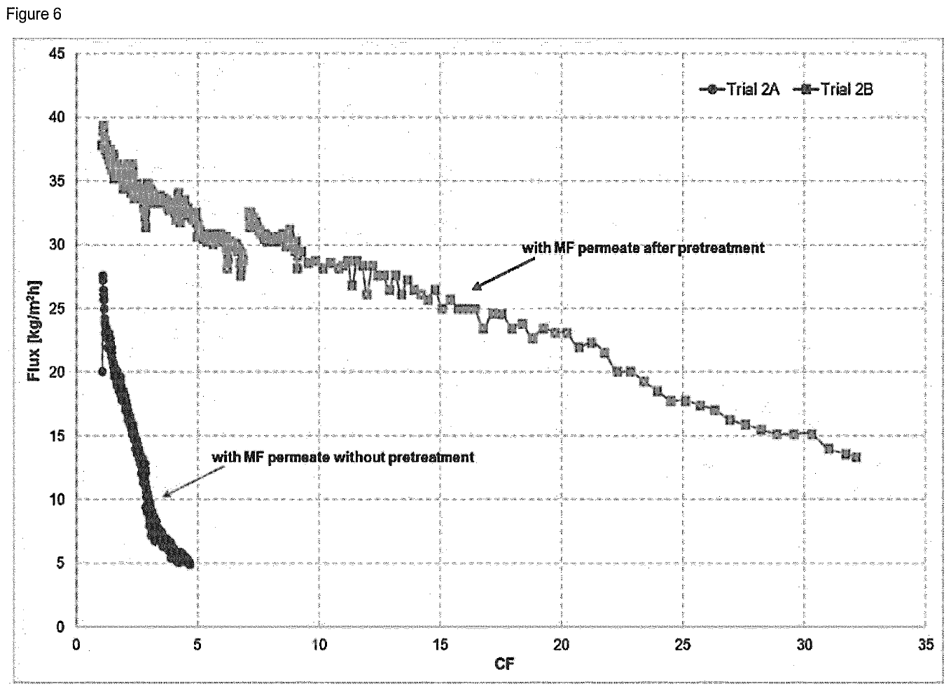

FIG. 6 provides trends of flux for both trials as a function of concentration factor. Membrane performance in trial 2B was still 2.5 times higher after CF of 39 as in trial 2A after CF of 2.9. After each restart the flux increased but relatively quick reached the previous values.

### Summary: Advantages of Pre-Treatment

Biomass Separation:
70% higher flux
15% lower pressure difference through membrane length
15-time less proteins in permeate
3-time lower APHA
At least 10% higher vanillin yield in permeate

UF Separation of Macromolecules:
13-time higher concentration factor of the feed
5-time higher average flux
By factor 2 lower APHA
19-time less proteins in permeate
Significantly higher vanillin yield (97% vs 67%)

### Example 3

In this example the dependence of active carbon (AC) concentration on vanillin separation was investigated. AC concentration was adjusted to 0.7% in trial 3A and 1.5% in trial 3B. Additionally the procedure for biomass separation was modified: In the first membrane filtration, after DF=5 an additional CF step was conducted until reaching dead volume of the unit (CF in trail 3A=2.65, in trial 3B=2.71).

The procedure for the UF step did not changed.

Results from Separation of Biomass from Dissolved Components (MF)

Table 13 presents the performance data of biomass separation with ceramic membrane for fermentation broth with two AC concentrations: Trial 3A: AC=0.7% and Trial 3B: AC=1.5%. Membrane performance is similar in both trials indicating that already 0.7% AC significantly improves the membrane performance. (Compare the results from example 1 and 2).

TABLE 13
Performance data

| | AC | Flux | DP | cross-flow |
|---|---|---|---|---|
| Trial | [%] | [kg/m²h] | [bar] | [m/s] |
| 3A | 0.7 | 232 | 0.86 | 3.5 |
| 3B | 1.5 | 247 | 0.85 | 3.5 |

Table 14 presents the membrane regeneration data for both trials. Slightly better performance was obtained in trial 3B.

TABLE 14
Membrane regeneration data

| | | | | | Flux- |
|---|---|---|---|---|---|
| | Flux [kg/m²h] | | | Regeneration | Improvement |
| Trial | before trial | after trial | after CIP | [%] | [%] |
| 3A | 412 | 190 | 395 | 96 | |
| 3B | 421 | 205 | 459 | 109 | 8 |

Table 15 (see following page) presents mass balance for both trials for APHA, protein, dry content of dissolved components, and vanillin. The data show clearly that pretreatment with higher AC concentration (trial 3B with 1.5% AC) much better reduces color components. The concentration of color components (as APHA) drops in the pretreated fermentation broth already by factor 4 when using 0.7% AC and by factor 12 when adding 1.5% AC. Further color reduction is reached during MF process, when using 1.5% AC an APHA value in permeate is two times lower than when using 0.7% AC.

Due to the pre-treatment (acidic conditions and AC) the concentration of dissolved proteins drops in the pretreated fermentation broth already by a factor 8 independently of the AC concentration. Further protein reduction takes place during MF process resulting in ca. 50% protein reduction in permeate when using 0.7% AC and in ca. 70% protein reduction when using 1.5% AC.

TABLE 15
Mass balance of both trials

| | | Mass | | Protein | | Rₚᵣₒₜₑᵢₙ | DC (dissolved) | | Vanillin | | | R_{Vanillin} |
|---|---|---|---|---|---|---|---|---|---|---|---|---|
| Trial | Stream | [g] | APHA | [mg/l] | [g] | [%] | [%] | [g] | [g/l] | [g] | Yield | [%] |
| 3A | Ferm. broth | 1132.0 | 5837 | 648 | 0.73 | | | | 9.1 | 10.3 | | |
| | Feed pretr. | 1148.3 | 1416 | 81 | 0.09 | | 7.83 | 89.9 | 7.4 | 8.5 | | |
| | Permeate | 6436.4 | 214 | 5.6 | 0.04 | 82 | 1.34 | 86.2 | 1.5 | 9.7 | >98 | |
| | Retentate | 452.3 | 24 | 95 | 0.04 | | 0.09 | 0.4 | <0.5 | <0.2 | <0.2 | <26 |
| 3B | Ferm. broth | 1187.0 | 5837 | 648 | 0.77 | | | | 9.1 | 10.8 | | |
| | Feed pretr. | 1200.7 | 487 | 80 | 0.10 | | 7.54 | 90.5 | 4.4 | 5.3 | | |
| | Permeate | 6751.8 | 107 | 3.9 | 0.03 | 95 | 1.28 | 86.4 | 1.6 | 10.8 | >98 | |
| | Retentate | 457.5 | 4 | 65 | 0.03 | | 0.08 | 0.4 | <0.5 | <0.2 | <0.2 | <39 |

Yield is given as % (w/w); R_{Protein} is the retention of proteins, R_{Vanillin} is the retention of Vanillin

During the DF step proteins and color components have not been redissolved and even their concentration was further reduced, resulting in much cleaner permeate.

Vanillin in both retentates was below detection limit, indicating that the membrane procedure consisting of DF=5 followed by CF=2.5 reaches vanillin yield in permeate of >98%. Such high yield was already observed in previous examples. Nevertheless, AC adsorbs vanillin, vanillin is washed out during DF step with demineralized water as diafiltration medium.

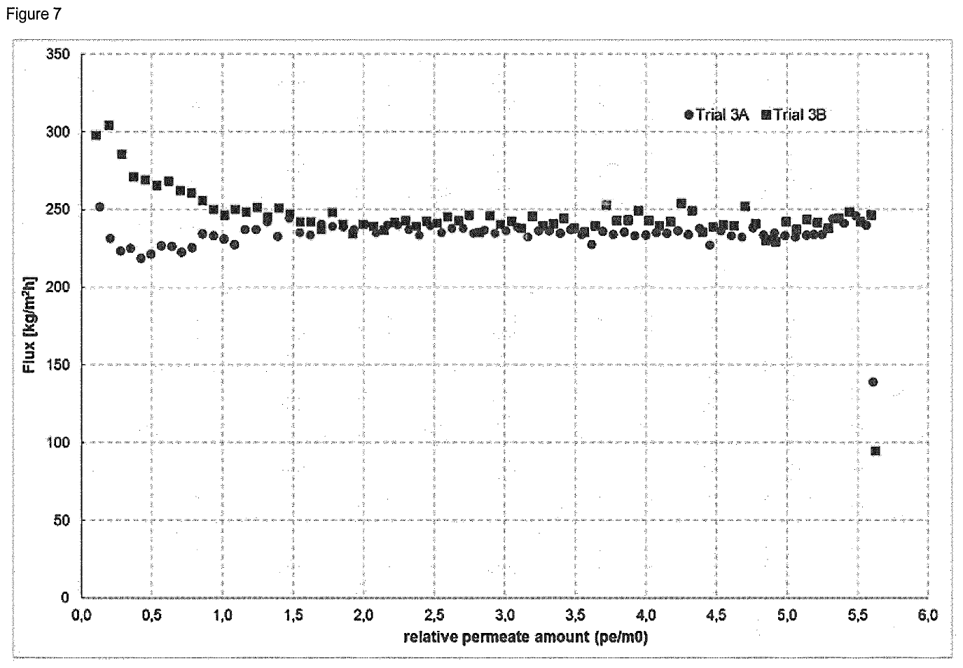

FIG. 7 presents flux trends as a function of relative permeate amount for both trials. The trends indicate that both fluxes are practically equal, and that higher AC concentration did not result in higher flux. During the final concentration step, which begins when reaching relative permeate amount of 4 the flux remains constant.

### SUMMARY

Fermentation broth pretreatment (acidification and AC addition) results in higher flux. However, no significant difference in membrane performance was observed in both trials with 0.7 and 1.5% AC
Pretreatment is very effective for reduction of color components and proteins present in the fermentation broth
Reduction of color (APHA) increases with increasing AC concentration
Reduction of proteins is similar for both AC concentrations
A cross flow filtration (MF) additionally allows for reduction of color and proteins membrane
Even if vanillin adsorbs on AC, it is possible to reach almost 100% vanillin yield in permeate. By proper adjusting of diafiltration and concentration steps the amount of permeate can be reduced.

### CITED PUBLICATIONS

CN105132472
CN105219806
EP 2 379 708
EP1081212
EP2583744
KR10-1163542
US20110028759
U.S. Pat. No. 6,133,033—CN105132472,
U.S. Pat. No. 9,115,377
Vandamme & Soetart, Journal of Chemical Technology and Biotechnology 77:1323-1332 2002
WO2007099230
WO2015002528
WO2020/223417
WO2020/223418

1.-15. (canceled)

16. A method for separating biomass from a solution comprising biomass and at least one aroma compound, comprising the following steps in this order:
(1) providing the solution comprising biomass and one or more aroma compound(s);
(2) setting the pH value of the solution below 7.0 if needed, preferably by adding at least one acid to the solution comprising biomass and the at least one aroma compound;
(3) adding at least one adsorbing agent to the solution comprising biomass and aroma compounds; and
(4) carrying out a first membrane filtration so as to separate the biomass from the solution comprising at least one aroma compound wherein the at least one aroma compound has one glycosidic bond or no glycosidic bonds and is not a protein.

17. The method according to claim 16, wherein the pH value of the solution is lowered to a pH value in the range of 3.0 to 5.5.

18. The method according to claim 16, wherein said at least one acid is an acid selected from the group consisting of H₂SO₄, H₃PO₄, HCl, HNO₃ and CH₃CO₂H.

19. The method according to claim 16, wherein said adsorbing agent is added in an amount in the range of 0.3% to 3% by weight.

20. The method according to claim 16, wherein said adsorbing agent is added as a powder having a particle size distribution with a diameter d50 in the range of 2 μm to 25 μm.

21. The method according to claim 16, wherein said first membrane filtration is carried out as cross-flow microfiltration or cross-flow ultrafiltration.

22. The method according to claim 21, wherein said cross-flow microfiltration or cross-flow ultrafiltration includes a cross-flow speed in the range of 0.5 m/s to 6.0 m/s.

23. The method according to claim 21, wherein said cross-flow speed is equal to or below 3 m/s.

24. The method according to claim 16, wherein said first membrane filtration is carried out at a temperature of the solution in the range of 8° C. to 55° C.

25. The method according to claim 16, wherein said first membrane filtration is carried out by means of a ceramic microfiltration or ultrafiltration membrane having a pore size in the range of 20 nm to 800 nm, or wherein said first membrane filtration is carried out by means of a polymeric microfiltration membrane or polymeric ultrafiltration membrane having a cut-off in the range of 10 kDa to 200 nm.

26. The method according to claim 16, further comprising carrying out a second membrane filtration with the solution comprising aroma compounds obtained by the first membrane filtration, and optionally followed by a reverse osmosis.

27. The method according to claim 26, wherein said second membrane filtration is carried out at a temperature of the solution being in the range of 5° C. to 15° C.

28. The method according to claim 16, wherein said at least one aroma compound comprises at least one polar aroma compound selected from the group consisting of Ambrox, Ambrox-1,4-diol, furaneol, benzoic acid, phenylethanol, raspberry ketone, pyrazines, sclareol, vanillin, vanillyl alcohol and vanilla glycoside.

29. A processing unit comprising i) a vessel filled with a solution containing biomass, at least one adsorbing agent and at least one aroma compound, wherein the at least one aroma compound has one glycosidic bond or no glycosidic bonds, and is not a protein, and wherein the pH value of the solution is below 7.0; ii.) a first filtration membrane which is a microfiltration or an ultrafiltration membrane; iii.) means to carry out a first membrane filtration across said first filtration membrane, which is a microfiltration or ultra-filtration, to generate a permeate containing the bulk of the aroma compounds, wherein the processing unit further comprises means to have the solution at a temperature in the range of 8° C. to 55° C. during the first membrane filtration; and iv.) means to separate the permeate of the first membrane filtration from the solution as described in i). above; v.) means to transport said permeate of the first membrane filtration to a second filtration membrane, vi.) means to adjust the temperature of the permeate to a temperature below 20° C., vii.) a second filtration membrane, viii.) means to carry out a second membrane filtration at a temperature below 20° C., and ix.) means to keep separate the permeate of the second membrane filtration from the permeate of the first membrane filtration; and optionally x) means to carry out a reverse osmosis treatment of the permeate of the second membrane filtration;
wherein the surfaces of the parts of the processing unit that are in contact with the solution or any of the permeates are tolerant to pH values as low as pH 3.5 and optionally are made of material suitable for the production of food grade material.

30. A method for reducing wear and tear on and/or energy consumption of membrane filtration equipment used in the separation of biomass from a solution comprising at least one aroma compound, wherein the method comprises these steps in the following order:
i. providing the solution comprising biomass and aroma compound(s),
ii. adjusting the pH value of the solution to a pH value in the range of 3.0 to 5.5,
iii. adding one or more adsorbing agents to the solution comprising biomass and aroma compound(s);
iv. optionally an incubation step sufficient for the one or more adsorbing agents to bind the color components in the solution, and
v. carrying out first membrane filtration so as to separate the biomass from the solution comprising the comprising at least one aroma compound at cross-flow speeds of no more than 3 m/s.
vi. optionally carrying out a second membrane filtration with the solution comprising aroma compounds obtained by the first membrane filtration.
